(19) United States
(12) Patent Application Publication
(10) Pub. No.: US 2026/0192206 A1
(43) Pub. Date: Jul. 9, 2026

(54) ACTION CONTROL SYSTEM

(71) Applicant: SOFTBANK GROUP CORP., Tokyo (JP)

(72) Inventor: Masayoshi SON, Tokyo (JP)

(73) Assignee: SOFTBANK GROUP CORP., Tokyo (JP)

(21) Appl. No.: 19/464,652

(22) Filed: Jan. 30, 2026

Related U.S. Application Data
(63) Continuation of application No. PCT/JP2024/027593, filed on Aug. 1, 2024.

(30) Foreign Application Priority Data
Aug. 2, 2023 (JP) 2023-126183
Aug. 4, 2023 (JP) 2023-128191
Aug. 7, 2023 (JP) 2023-128897
Aug. 9, 2023 (JP) 2023-130313
Aug. 14, 2023 (JP) 2023-131827
Aug. 14, 2023 (JP) 2023-131828
Aug. 14, 2023 (JP) 2023-131846
Aug. 14, 2023 (JP) 2023-131924
Aug. 16, 2023 (JP) 2023-132499

(51) Int. Cl. A63H 3/00; A61B 5/00; A61B 5/16; A63H 3/02; A63H 3/28; A63H 11/00; G06V 40/10; G06V 40/20; G10L 15/22; G10L 25/66; G16H 20/10; G16H 40/67; H04R 1/02

(52) U.S. Cl. CPC A63H 3/003; A61B 5/165; A61B 5/486; A61B 5/7282; A61B 5/741; A61B 5/744; A63H 3/02; A63H 3/28; A63H 11/00; G06V 40/10; G06V 40/28; G10L 15/22; G10L 25/66; G16H 20/10; G16H 40/67; A63H 2200/00; H04R 1/028

(57) ABSTRACT

In an action control system, an action of an avatar includes producing and playing music in consideration of an event of a previous day, and in a case in which the action determination unit determines to produce and play music in consideration of the event of the previous day as the action of the avatar, the action determination unit acquires a summary of event data of the previous day stored in the history data and produces music based on the summary.

### CROSS-REFERENCE TO RELATED APPLICATIONS

This application is a continuation of International Application No. PCT/JP2024/027593, filed on Aug. 1, 2024, which claims priority from Japanese Patent Application No. 2023-126183, filed on Aug. 2, 2023, Japanese Patent Application No. 2023-128191, filed on Aug. 4, 2023, Japanese Patent Application No. 2023-128897, filed on Aug. 7, 2023, Japanese Patent Application No. 2023-130313, filed on Aug. 9, 2023, Japanese Patent Application No. 2023-131827, filed on Aug. 14, 2023, Japanese Patent Application No. 2023-131828, filed on Aug. 14, 2023, Japanese Patent Application No. 2023-131846, filed on Aug. 14, 2023, Japanese Patent Application No. 2023-131924, filed on Aug. 14, 2023, and Japanese Patent Application No. 2023-132499, filed on Aug. 16, 2023. The entire disclosure of each of the above applications is incorporated herein by reference.

### BACKGROUND

### Technical Field

The present invention relates to an action control system.

### Background Art

Japanese Patent No. 6053847 discloses a technique for determining an appropriate action of a robot for a state of a user. In the related art of Patent Literature 1, in a case in which a robot has recognized a user's reaction in a case in which the robot executed a specific action and an action of the robot in response to the recognized user's reaction has not been determined, the action of the robot is updated by receiving information regarding the action suitable for the user's recognized state from a server.

### SUMMARY OF INVENTION

### Technical Problem

However, in the related art, there is room for improvement in causing the robot to execute an appropriate action for the user's action.

### Solution to Problem

According to a first aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that stores event data including an emotion value determined by the emotion determination unit and data including the action of the user in history data; and an action control unit that displays the avatar in an image display area of the electronic equipment, in which the avatar actions include producing and playing music in consideration of an event of a previous day, and in a case in which the action determination unit determines, as an action of the avatar, to produce and play music in consideration of an event of a previous day, the action determination unit acquires a summary of event data of the previous day stored in the history data and produces music based on the summary.

According to a second aspect of the invention, the action determination model is a data generation model capable of generating data according to input data, and the action determination unit inputs data indicating at least one of the user state, the state of the electronic equipment, the emotion of the user, or the emotion of the avatar, together with data for asking about an avatar action to the data generation model, and determines an action of the avatar based on an output of the data generation model.

According to a third aspect of the invention, in a case in which the action determination unit determines to produce and play music in consideration of an event on the previous day, as an action of the avatar, the action control unit is caused to control the avatar such that the avatar plays the music.

According to a fourth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that stores event data including an emotion value determined by the emotion determination unit and data including the action of the user in history data; and an action control unit that displays the avatar in an image display area of the electronic equipment, in which the avatar actions include outputting advice information for utterance of the user in a meeting, and in a case in which a summary of minutes of a past meeting is acquired and an utterance in a predetermined relationship with the summary is made, the action determination unit determines, as an action of the avatar, to output the advice information for the utterance of the user in the meeting, and outputs the advice information according to a content of the utterance.

According to a fifth aspect of the invention, the action determination model is a data generation model capable of generating data according to input data, and the action determination unit inputs data indicating at least one of the user state, the state of the electronic equipment, the emotion of the user, or the emotion of the avatar, together with data for asking about an avatar action to the data generation model, and determines an action of the avatar based on an output of the data generation model.

According to a sixth aspect of the invention, in a case in which the action determination unit determines, as an action of the avatar, to output the advice information for the utterance of the user in the meeting, the action determination unit operates the avatar so as to determine a conversation to utter further based on a state of the electronic equipment of another user or an emotion of another avatar displayed on the electronic equipment of the other user.

According to a seventh aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that stores event data including an emotion value determined by the emotion determination unit and data including the action of the user in history data; and an action control unit that displays the avatar in an image display area of the electronic equipment, in which the avatar actions include outputting a summary of an event of a previous day in an utterance or a gesture, in a case in which the action determination unit determines, as an action of the avatar, to output a summary of an event of a previous day in an utterance or a gesture, the action determination unit acquires a summary of event data of the previous day stored in the history data when a conversation or a gesture predetermined by the user is detected, and the action control unit controls the avatar to output the summary through an utterance or a gesture.

According to an eighth aspect of the invention, the action determination model is a data generation model capable of generating data according to input data, and the action determination unit adds a fixed sentence instructing to summarize the event of the previous day to a text representing the event data of the previous day, inputs the text to the data generation model, and generates the summary based on an output of the data generation model.

According to a ninth aspect of the invention, the conversation or gesture predetermined by the user is a conversation in which the user tries to remember the event of the previous day or a gesture in which the user thinks about something.

According to a tenth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that stores event data including an emotion value determined by the emotion determination unit and data including the action of the user in history data; and an action control unit that displays the avatar in an image display area of the electronic equipment, in which the avatar actions include reflecting an event of a previous day in an emotion of the next day, in a case in which the action determination unit determines, as an action of the avatar, to reflect an event of a previous day in an emotion of the next day, the action determination unit acquires a summary of event data of the previous day stored in the history data and determines an emotion to be held on the next day based on the summary, and the action control unit controls the avatar to express the emotion to be held on the next day.

According to an eleventh aspect of the invention, the action determination model is a data generation model capable of generating data according to input data, and the action determination unit adds a fixed sentence instructing to summarize the event of the previous day to a text representing the event data of the previous day, inputs the text to the data generation model, generates the summary based on an output of the data generation model, adds a fixed sentence for asking about the emotion to have on the next day to a text representing the summary, inputs the text to the data generation model, and determines the emotion to have on the next day based on an output of the data generation model.

According to a twelfth aspect of the invention, the summary includes information indicating an emotion of the previous day, and the emotion to be held on the next day will be carried over from the emotion of the previous day.

According to a thirteenth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at predetermined timing to determine any of multiple types of avatar actions including not acting as an action of the avatar; a memory control unit that stores event data including an emotion value determined by the emotion determination unit and data including the action of the user in history data; and an action control unit that displays the avatar in an image display area of the electronic equipment, in which the avatar actions include performing support for progress of a meeting for the user in the meeting, and in a case in which the meeting is in a predetermined state, the action determination unit determines to output support for the progress of the meeting for the user in the meeting and outputs support for the progress of the meeting, as an action of the avatar.

According to a fourteenth aspect of the invention, the action determination model is a data generation model capable of generating data according to input data, and the action determination unit inputs data indicating at least one of the user state, the state of the electronic equipment, the emotion of the user, or the emotion of the avatar, together with data for asking about an avatar action to the data generation model, and determines an action of the avatar based on an output of the data generation model.

According to a fifteenth aspect of the invention, in a case in which the action determination unit determines, as an action of the avatar, to output support for the progress of the meeting for the user in the meeting, the action determination unit causes the avatar to operate to determine a content to support the progress further based on a state of the electronic equipment of another user or an emotion of another avatar displayed on the electronic equipment of the other user.

According to a sixteenth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that determines an action of the avatar based on at least one of the user state, the state of the electronic equipment, the emotion of the user, or the emotion of the avatar; and an action control unit that displays the avatar in an image display area of the electronic equipment, in which in a case in which the action determination unit determines to take minutes of a meeting as an action of the avatar, the action determination unit acquires a speech content of the user by voice recognition, identifies a speaker through voiceprint authentication, and acquires an emotion of the speaker based on a determination result of the emotion determination unit, and creates minutes data representing a combination of the speech content of the user, an identification result of the speaker, and the emotion of the speaker.

According to a seventeenth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; a memory control unit that stores event data including an emotion value determined by the emotion determination unit and data including the action of the user in history data; an action determination unit that uses at least one of the user state, the state of the electronic equipment, the emotion of the user, or the emotion of the avatar, together with a summary image visualizing a content of a summary sentence that is a sentence about a history of a previous day of the user represented by the history data and an action determination model at a predetermined timing to determine any of multiple types of avatar actions including not acting, as an action of the avatar; and an action control unit that displays the avatar in an image display area of the electronic equipment, in which the avatar actions include an action related to an action history of the user represented by the summary image, and in a case in which the action determination unit determines to utter a topic about the action history of the user as an action of the avatar, the action determination unit determines to utter a topic about a state of the user estimated from the action history of the user.

According to an eighteenth aspect of the invention, an action control system is provided. The action control system includes a state recognition unit that recognizes a user state including an action of a user and a state of electronic equipment; an emotion determination unit that determines an emotion of the user or an emotion of an avatar representing an agent for interacting with the user; a memory control unit that stores event data including an emotion value determined by the emotion determination unit and data including the action of the user in history data; an action determination unit that uses at least one of the user state, the state of the electronic equipment, the emotion of the user, or the emotion of the avatar, together with a summary sentence of a history of a previous day of the user created from history data of the previous day of the user stored in the memory control unit, and an action determination model at a predetermined timing to determine any of multiple types of avatar actions including not acting, as an action of the avatar; and an action control unit that displays the avatar in an image display area of the electronic equipment, in which the avatar actions include an action related to an action history of the user represented by the summary sentence, and in a case in which the action determination unit determines to utter a topic about the action history of the user as an action of the avatar, the action determination unit determines to utter a topic about a state of the user.

According to a nineteenth aspect of the invention, an action control system is provided. The action control system includes an input unit that receives a user input; a processing unit that performs a specific process using a sentence generation model that generates a sentence corresponding to input data; and an output unit that causes an avatar representing an agent for interacting with a user to be displayed in an image display area of electronic equipment so as to output a result of the specific process, in which an action of the avatar from the output unit includes acquiring and outputting a response regarding a presentation content in a meeting held by the user, and the processing unit determines whether a condition for a presentation content in the meeting is satisfied as a predetermined trigger condition, and in a case in which the trigger condition is satisfied, the processing unit acquires and outputs a response to the presentation content in the meeting as a result of the specific process by using an output of the sentence generation model when at least an email description item, a schedule table description item, or a meeting speech item obtained from a user input in a specific period is set as the input data.

According to a twentieth aspect of the invention, the electronic equipment is a headset-type terminal.

According to a twenty-first aspect of the invention, the electronic equipment is an eyeglass-type terminal.

### BRIEF DESCRIPTION OF DRAWINGS

FIG. 1 schematically illustrates an example of a system 5 according to a first embodiment.

FIG. 2A schematically illustrates a functional configuration of a robot 100 according to the first embodiment.

FIG. 2B schematically illustrates another functional configuration of the robot 100 according to the first embodiment.

FIG. 2C schematically illustrates a functional configuration of a specific processing unit of the robot 100 according to the first embodiment.

FIG. 3 schematically shows an example of an operation flow of a collecting process by the robot 100 according to the first embodiment.

FIG. 4A schematically shows an example of an operation flow of a response process by the robot 100 according to the first embodiment.

FIG. 4B schematically shows an example of an operation flow of an autonomous process by the robot 100 according to the first embodiment.

FIG. 4C schematically shows an example of an operation flow of a specific process by the robot 100 according to the first embodiment.

FIG. 5 illustrates an emotion map 400 on which multiple emotions are mapped.

FIG. 6 illustrates an emotion map 900 on which multiple emotions are mapped.

FIG. 7(A) is an external view of a stuffed toy 100N according to a second embodiment, and FIG. 7(B) is an internal structural view of the stuffed toy 100N.

FIG. 8 is a rear front view of the stuffed toy 100N according to the second embodiment.

FIG. 9 schematically illustrates a functional configuration of the stuffed toy 100N according to the second embodiment.

FIG. 10 schematically illustrates a functional configuration of an agent system 500 according to a third embodiment.

FIG. 11 illustrates an example of an operation of the agent system.

FIG. 12 illustrates an example of an operation of the agent system.

FIG. 13 schematically illustrates a functional configuration of an agent system 700 according to a fourth embodiment.

FIG. 14 illustrates an example of a usage mode of the agent system using smart glasses.

FIG. 15 schematically illustrates a functional configuration of an agent system 800 according to a fifth embodiment.

FIG. 16 illustrates an example of a headset-type terminal.

FIG. 17 schematically illustrates an example of a hardware configuration of a computer 1200.

### DESCRIPTION OF EMBODIMENTS

Hereinafter, the invention will be described through embodiments of the invention, and the following embodiments do not limit the invention according to the claims. In addition, not all combinations of features described in the embodiments are essential to the solution of the invention.

### First Embodiment

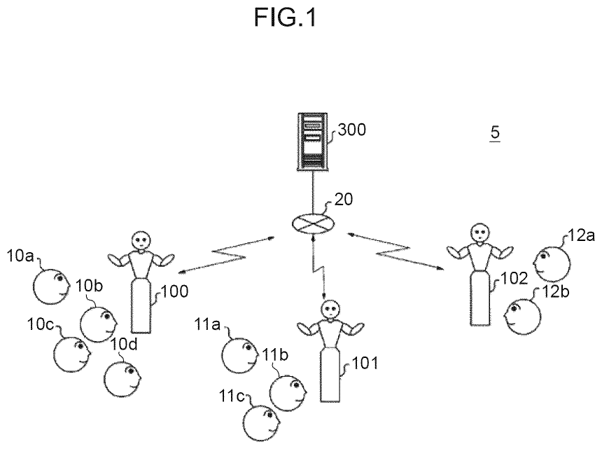

FIG. 1 schematically illustrates an example of a system 5 according to the present embodiment. The system 5 includes a robot 100, a robot 101, a robot 102, and a server 300. A user 10a, a user 10b, a user 10c, and a user 10d are users of the robot 100. A user 11a, a user 11b, and a user 11c are users of the robot 101. A user 12a and a user 12b are users of the robot 102. Note that, in the description of the present embodiment, the user 10a, the user 10b, the user 10c, and the user 10d may be collectively referred to as “user 10”. Furthermore, the user 11a, the user 11b, and the user 11c may be collectively referred to as “user 11”. Furthermore, the user 12a and the user 12b may be collectively referred to as “user 12”. The robot 101 and the robot 102 have substantially the same functions as those of the robot 100. Thus, the system 5 will be described focusing on the functions of the robot 100.

The robot 100 has conversations with the user 10 and provides videos to the user 10. At this time, the robot 100 performs a conversation with the user 10 and provides a video to the user 10, and the like in cooperation with the server 300 and the like that can communicate via a communication network 20. For example, the robot 100 not only learns an appropriate conversation by itself, but also performs learning so that a conversation with the user 10 can be advanced more appropriately in cooperation with the server 300. Further, the robot 100 causes the server 300 to record captured video data and the like of the user 10, requests the server 300 for the video data and the like if necessary, and provides the video data and the like to the user 10.

Furthermore, the robot 100 has an emotion value indicating the type of its own emotion. For example, the robot 100 has emotion values indicating the intensity of each emotion such as “joy”, “anger”, “sorrow”, “pleasure”, “comfort”, “discomfort”, “relief”, “anxiety”, “sadness”, “excitement”, “worry”, “reassurance”, “fulfillment”, “emptiness”, and “neutral”. For example, in a case in which the robot 100 has a conversation with the user 10 with a high emotion value of excitement, the robot emits voice at a fast speed. As described above, the robot 100 can express its own emotion by action.

Furthermore, the robot 100 may be configured to determine an action of the robot 100 corresponding to an emotion of the user 10 by matching a sentence generation model using artificial intelligence (AI) with an emotion engine. Specifically, the robot 100 may be configured to recognize an action of the user 10, determine the emotion of the user 10 for the action of the user, and determine an action of the robot 100 corresponding to the determined emotion.

More specifically, in a case in which the robot 100 has recognized an action of the user 10, the robot 100 automatically generates the action content to be taken by the robot 100 in response to the action of the user 10 by using a preset sentence generation model. The sentence generation model may be interpreted as an algorithm and an arithmetic operation for an automatic interaction process based on characters. Since the sentence generation model is known as disclosed in, for example, Japanese Patent Application Laid-Open (JP-A) No. 2018-081444 and ChatGPT (retrieved from the Internet <URL: https://openai.com/blog/chatgpt>), detailed description thereof will be omitted. Such a sentence generation model is configured by a large-scale language model (LLM).

As described above, in the present embodiment, it is possible to reflect the emotions of the user 10 and the robot 100 and various linguistic information in actions of the robot 100 by combining the large-scale language model and the emotion engine. That is, according to the present embodiment, synergistic effects can be obtained by combining the sentence generation model and the emotion engine.

Further, the robot 100 has the function of recognizing actions of the user 10. The robot 100 recognizes actions of the user 10 by analyzing face images of the user 10 acquired by the camera function and voices of the user 10 acquired by the microphone function. The robot 100 determines an action to be performed by the robot 100 based on a recognized action of the user 10 or the like.

As an example of an action determination model, the robot 100 stores a rule for defining an action to be performed by the robot 100 based on an emotion of the user 10, an emotion of the robot 100, and an action of the user 10, and performs various actions according to the rule.

Specifically, the robot 100 includes, as an example of the action determination model, reaction rules for determining an action of the robot 100 based on an emotion of the user 10, an emotion of the robot 100, and an action of the user 10. According to the reaction rules, for example, in a case in which an action of the user 10 is “laughing”, the action of the robot 100 is set to “laughing”. In addition, according to the reaction rules, in a case in which an action of the user 10 is “getting angry”, the action of the robot 100 is set to “apologizing”. In addition, according to the reaction rules, in a case in which an action of the user 10 is “asking a question”, the action of the robot 100 is set to “answering”. According to the reaction rules, in a case in which an action of the user 10 is “expressing sadness”, the action of the robot 100 is set to “showing encouragement”.

In a case in which the robot 100 recognizes the action of the user 10 as “getting angry” based on the reaction rules, the robot chooses the action of “apologizing” defined in the reaction rules as an action to be performed by the robot 100. For example, in the case of choosing the action of “apologizing”, the robot 100 performs the action of “apologizing” and outputs a voice expressing a word of “apology”.

Furthermore, in a case in which a condition that the emotion of the robot 100 is “neutral” (that is, “joy”=0, “anger”=0, “sadness”=0, and “pleasure”=0) and the state of the user 10 is “being alone is lonely” is satisfied, it is defined that the content of emotion change in the emotion of the robot 100 to “worried” and the action of “showing encouragement” can be performed.

In a case in which the robot 100 recognizes that the current emotion of the robot 100 is “neutral” and the user 10 is alone and feels sad based on the reaction rules, the emotion value of “sorrow” of the robot 100 is increased. Furthermore, the robot 100 selects an action of “showing encouragement” defined in the reaction rule as an action to be performed on the user 10. For example, in a case in which the action of “showing encouragement” is selected, the robot 100 converts the phrase “What's wrong?” expressing concern into a voice expressing concern, and outputs the voice.

Furthermore, the robot 100 transmits, to the server 300, user reaction information indicating that a positive reaction has been obtained from the user 10 due to this action. The user reaction information includes, for example, a user action of “getting angry”, an action of the robot 100 of “apologizing”, a positive reaction of the user 10, and an attribute of the user 10.

The server 300 stores the user reaction information received from the robot 100. Note that the server 300 receives the user reaction information not only from the robot 100 but also from each of the robot 101 and the robot 102 and stores the user reaction information. Then, the server 300 analyzes the user reaction information from the robot 100, the robot 101, and the robot 102, and updates the reaction rules.

The robot 100 inquires the server 300 about the updated reaction rules to receive the updated reaction rules from the server 300. The robot 100 incorporates the updated reaction rules into the reaction rules stored in the robot 100. As a result, the robot 100 can incorporate the reaction rules acquired by the robot 101, the robot 102, and the like into its own reaction rules.

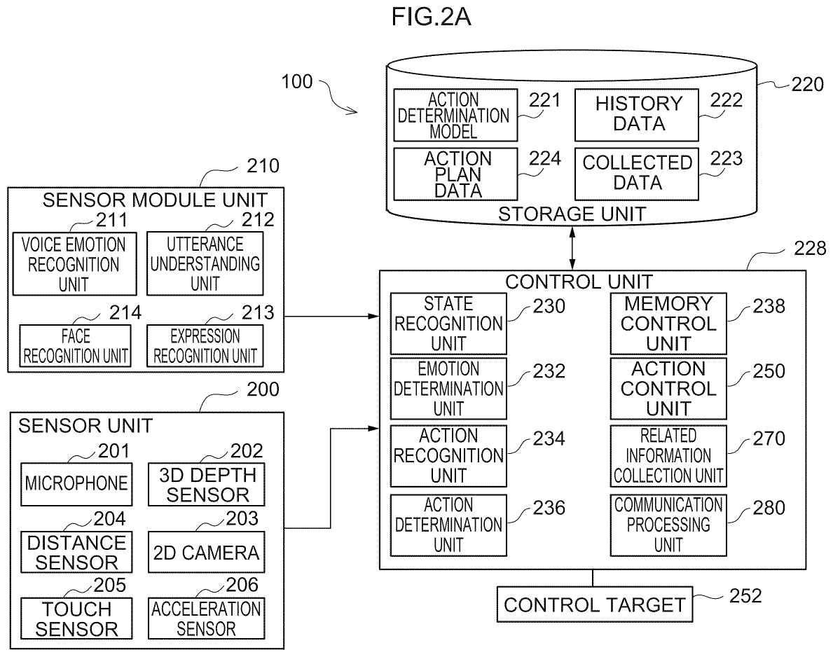

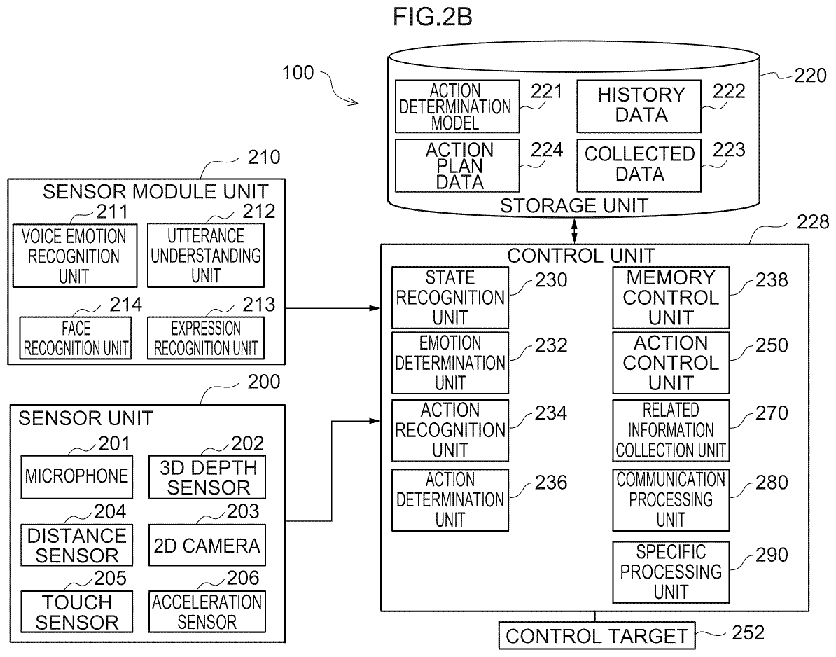

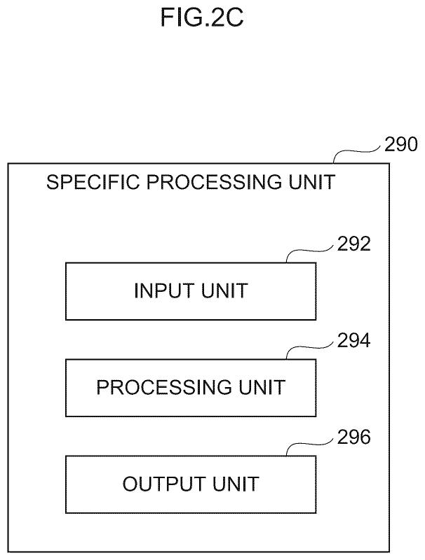

FIG. 2A schematically illustrates a functional configuration of the robot 100. The robot 100 includes a sensor unit 200, a sensor module unit 210, a storage unit 220, a control unit 228, and a control target 252. The control unit 228 includes a state recognition unit 230, an emotion determination unit 232, an action recognition unit 234, an action determination unit 236, a memory control unit 238, an action control unit 250, a related information collection unit 270, and a communication processing unit 280. Note that, as illustrated in FIG. 2B, the robot 100 may further include a specific processing unit 290.

The control target 252 includes a display device, a speaker, an LED at the eye part, motors that drive arms, hands, feet, and the like. Postures and gestures of the robot 100 are controlled by controlling motors for arms, hands, and feet. Some of the emotions of the robot 100 can be expressed by controlling these motors. Furthermore, expressions of the robot 100 can be represented by controlling light emission states of the LEDs at the eye part of the robot 100. Note that the postures, gestures, and expressions of the robot 100 are examples of attitudes of the robot 100.

The sensor unit 200 includes a microphone 201, a 3D depth sensor 202, a 2D camera 203, a distance sensor 204, a touch sensor 205, and an acceleration sensor 206. The microphone 201 continuously detects sound and outputs voice data. Note that the microphone 201 may be provided on the head of the robot 100 and may have a function of performing binaural recording. The 3D depth sensor 202 detects outlines of an object by continuously emitting an infrared pattern and analyzing the infrared pattern from an infrared image continuously captured by an infrared camera. The 2D camera 203 is an example of an image sensor. The 2D camera 203 captures an image with visible light and generates image information from visible light. The distance sensor 204 detects a distance to an object by emitting, for example, a laser, an ultrasonic wave, or the like. Note that the sensor unit 200 may further include a clock, a gyro sensor, a sensor for motor feedback, and the like.

Note that, among the components of the robot 100 illustrated in FIG. 2A, the components other than the control target 252 and the sensor unit 200 are examples of the components included in the action control system of the robot 100. The control target 252 is a target to be controlled by the action control system of the robot 100.

The storage unit 220 includes an action determination model 221, history data 222, collected data 223, and action plan data 224. The history data 222 includes past emotion values of the user 10, past emotion values of the robot 100, and an action history, and specifically includes multiple pieces of event data including the emotion values of the user 10, the emotion values of the robot 100, and actions of the user 10. The data including the actions of the user 10 includes camera images representing the actions of the user 10. The emotion values and the action history are recorded for each user 10 by being associated with identification information of the user 10, for example. At least a part of the storage unit 220 is implemented by a storage medium such as a memory. A person DB that stores face images of the user 10, attribute information of the user 10, and the like may be included. Note that, among the components of the robot 100 illustrated in FIG. 2A, the functions of the components other than the control target 252, the sensor unit 200, and the storage unit 220 can be realized by a CPU operating according to programs. For example, the functions of these components can be implemented as operations of the CPU by basic software (OS) and programs operating on the OS.

The sensor module unit 210 includes a voice emotion recognition unit 211, an utterance understanding unit 212, an expression recognition unit 213, and a face recognition unit 214. Information detected by the sensor unit 200 is input to the sensor module unit 210. The sensor module unit 210 analyzes information detected by the sensor unit 200 and outputs the analysis result to the state recognition unit 230.

The voice emotion recognition unit 211 of the sensor module unit 210 analyzes a voice of the user 10 detected by the microphone 201 to recognize the emotion of the user 10. For example, the voice emotion recognition unit 211 extracts a feature such as a frequency component of the utterance and recognizes the emotion of the user 10 based on the extracted feature. The utterance understanding unit 212 analyzes the voice of the user 10 detected by the microphone 201 and outputs character information indicating the utterance content of the user 10.

The expression recognition unit 213 recognizes the facial expression of the user 10 and the emotion of the user 10 from an image of the user 10 captured by the 2D camera 203. For example, the expression recognition unit 213 recognizes the facial expression and emotion of the user 10 based on the shapes, positional relationships, and the like of the user's eyes and mouth.

The face recognition unit 214 recognizes the face of the user 10. The face recognition unit 214 recognizes the user 10 by matching a face image stored in the person DB (not illustrated) with a face image of the user 10 captured by the 2D camera 203.

The state recognition unit 230 recognizes the state of the user 10 based on the information analyzed by the sensor module unit 210. For example, analysis results of the sensor module unit 210 are used to perform processing mainly related to perception. For example, perceptual information such as “Dad is alone” and “There is a 90% probability that dad is not smiling” is generated. A process of understanding the meaning of the generated perceptual information is performed. For example, semantic information such as “Dad alone seems to be lonely” is generated.

The state recognition unit 230 recognizes the state of the robot 100 based on the information detected by the sensor unit 200. For example, the state recognition unit 230 recognizes the remaining battery level of the robot 100, the brightness of the surrounding environment of the robot 100, and the like as the states of the robot 100.

The emotion determination unit 232 determines an emotion value indicating the emotion of the user 10 based on the information analyzed by the sensor module unit 210 and the state of the user 10 recognized by the state recognition unit 230. For example, the information analyzed by the sensor module unit 210 and the recognized state of the user 10 are input to a pre-trained neural network to acquire an emotion value indicating the emotion of the user 10.

Here, the emotion value indicating the emotion of the user 10 is a value indicating whether the emotion of the user is positive or negative. For example, if the emotion of the user is a bright emotion accompanied with pleasure or comfort, such as “joy”, “pleasure”, “comfort”, “relief”, “excitement”, “reassurance”, and “fulfillment”, a positive value is indicated, and the value becomes greater as the emotion is brighter. If the user's emotion is an emotion that makes the user feel unpleasant, such as “anger”, “sorrow”, “discomfort”, “anxiety”, “sadness”, “worry”, and “emptiness”, a negative value is indicated, and the absolute value of the negative value increases as the user feels unpleasant. In a case in which the user's emotion is not any of the above (“neutral”), the value 0 is indicated.

Furthermore, the emotion determination unit 232 determines an emotion value indicating the emotion of the robot 100 based on the information analyzed by the sensor module unit 210, the information detected by the sensor unit 200, and the state of the user 10 recognized by the state recognition unit 230.

The emotion value of the robot 100 includes the emotion value for each of multiple emotion classifications, and is, for example, a value (0 to 5) indicating the intensity of each of “joy”, “anger”, “sorrow”, and “pleasure”.

Specifically, the emotion determination unit 232 determines an emotion value indicating the emotion of the robot 100 according to a rule for updating the emotion value of the robot 100 defined in association with the information analyzed by the sensor module unit 210 and the state of the user 10 recognized by the state recognition unit 230.

For example, in a case in which the state recognition unit 230 recognizes that the user 10 seems to be lonely, the emotion determination unit 232 increases the emotion value for “sorrow” of the robot 100. Furthermore, in a case in which the state recognition unit 230 recognizes that the user 10 has a smiling face, the emotion value for “joy” of the robot 100 is increased.

Note that the emotion determination unit 232 may determine the emotion value indicating the emotion of the robot 100 in further consideration of the state of the robot 100. For example, in a case in which the remaining battery level of the robot 100 is low, a case in which the surrounding environment of the robot 100 is completely dark, or the like, the emotion value for “sorrow” of the robot 100 may be increased. Furthermore, the emotion value for “anger” may be increased in a case in which the user 10 continuously talks even though the remaining battery level is low.

The action recognition unit 234 recognizes an action of the user 10 based on the information analyzed by the sensor module unit 210 and the state of the user 10 recognized by the state recognition unit 230. For example, the information analyzed by the sensor module unit 210 and the recognized state of the user 10 are input to a pre-trained neural network, the probability of each of multiple predetermined action classifications (for example, “smile”, “getting angry”, “asking”, and “getting sad”) is acquired, and the action classification having the highest probability is recognized as the action of the user 10.

As described above, in the present embodiment, the robot 100 acquires the utterance content of the user 10 after identifying the user 10, but in acquiring and using the utterance content, the action control system of the robot 100 according to the present embodiment considers protection of personal information and privacy of the user 10 in addition to acquiring necessary consent from the user 10 according to laws and regulations.

Next, processing of the action determination unit 236 when the robot 100 performs a response process in which the robot responds to the action of the user 10 will be described.

The action determination unit 236 determines an action corresponding to the action of the user 10 recognized by the action recognition unit 234 based on the current emotion value of the user 10 determined by the emotion determination unit 232, the history data 222 of the past emotion values determined by the emotion determination unit 232 before the current emotion value of the user 10 is determined, and the emotion value of the robot 100. In the present embodiment, a case in which the action determination unit 236 uses one most recent emotion value included in the history data 222 as a past emotion value of the user 10 will be described, but the disclosed technology is not limited to this aspect. For example, the action determination unit 236 may use multiple most recent emotion values as the past emotion values of the user 10, or may use emotion values that are earlier by a unit period such as one day earlier. Furthermore, the action determination unit 236 may determine an action corresponding to the action of the user 10 in further consideration of the history of the past emotion values of the robot 100 in addition to the current emotion value of the robot 100. The action determined by the action determination unit 236 includes a gesture performed by the robot 100 or utterance content of the robot 100.

The action determination unit 236 according to the present embodiment determines an action of the robot 100 based on a combination of the past emotion value and the current emotion value of the user 10, the emotion value of the robot 100, the action of the user 10, and the action determination model 221 as an action corresponding to the action of the user 10. For example, in a case in which the past emotion value of the user 10 is a positive value and the current emotion value is a negative value, the action determination unit 236 determines an action for positively changing the emotion value of the user 10 as an action corresponding to the action of the user 10.

In a case in which the action determination unit 236 determines to take meeting minutes as an action corresponding to the action of the user 10, the action determination unit acquires the speech content of the user 10 by voice recognition, identifies the speaker by voiceprint authentication, acquires the emotion of the speaker based on the determination result of the emotion determination unit 232, and creates minutes data representing a combination of the speech of the user 10, the identification result of the speaker, and the emotion of the speaker. The action determination unit 236 generates a summary of the text representing the minutes data by using a sentence generation model having an interaction function. The action determination unit 236 further generates a list of things that the user should do (to-do list) included in the summary by using the sentence generation model having an interaction function. This to-do list includes at least a person in charge (responsible person), an action content, and the deadline for each thing the user should do. The action determination unit 236 further transmits the minutes data, the summary, and the to-do list to the participants of the meeting. The action determination unit 236 further transmits a message to confirm things to do to the person in charge before the predetermined number of days determined by the deadline based on the person in charge and the deadline included in the list.

Specifically, when the user 10 speaks “take the minutes”, the action determination unit 236 determines to take the meeting minutes as an action corresponding to the action of the user 10. Thereby, the minutes data including the information indicating the person who spoke can be obtained. When the user 10 speaks “send the summary to the persons concerned” at the end of the meeting, the action determination unit 236 summarizes the meeting minutes, creates a to-do list, and transmits the summary and the to-do list to the persons concerned.

In the case of summarizing the meeting minutes, the text of the created minutes data and a fixed sentence “summarize the contents” are input to a generative AI that is a sentence generation model, and thereby a summary of the meeting minutes is acquired. Furthermore, when creating a to-do list, a text of the summary of meeting minutes and a fixed sentence “create a to-do list” are input to the generative AI that is a sentence generation model, and thereby a to-do list is acquired. As a result, upon understanding the content of the meeting, the meeting can be summarized, and thereby a to-do list can be created and the responsible parties for the to-do list can be organized. Categorization of the to-do list is performed by authenticating a voiceprint to recognize the person who spoke. Based on the determination result of the emotion determination unit 232, it is possible to combine the evaluation as to whether a person is reluctantly motivated to do something or is enthusiastically attempting to do something. It is possible to identify who will do what by when. In a case in which the person in charge, the deadline, and the like are not determined, speech to inquire with respect to the user 10 may be determined as an action of the robot 100. As a result, a message like “The person in charge for AAA has not been decided yet. Who would do it?” can be uttered by the robot 100.

Note that features related to the date and time may be extracted from the summary of the meeting minutes to register the features on a calendar or create a to-do list.

Furthermore, the action determination unit 236 may further determine, as the action of the robot 100, to speak the conclusion or summary of the meeting at the end of the meeting. In addition, the action determination unit 236 transmits the minutes data, the summary, and the to-do list to the participants of the meeting. The action determination unit 236 also sends a reminder of the to-do list to the person in charge.

As an example, in a case in which the action determination unit 236 determines to take the meeting minutes as an action corresponding to the action of the user 10, the action determination unit performs the processing of step 1 to step 9 below. (Step 1) Record the meeting proceedings contents.
(Step 2) Create minutes data from recorded data and summarize.
(Step 3) Identify who said what based on voiceprint authentication and determination of an emotion value by the emotion determination unit 232.
(Step 4) Create a to-do list of the meeting participants (because who had uttered what has been identified).
(Step 5) Register the to-do list on a calendar.
(Step 6) In a case in which a clear deadline is not determined, the meeting participants are asked that the to-do list is not completed, and asked again about missing information (5w1h) of the to-do list.
(Step 7) Complementarily specify emotion values of a person in charge of the to-do and the speaker at the time of creating the to-do list and reproducing the summary. Who has spoken with how much motivation and how much he/she is committed to do are visualized.
(Step 8) Transmit the meeting minutes to the meeting participants.
(Step 9) After the meeting, transmit a message to follow up on the items of the to-do list (follow up the deadline or the like).

In the reaction rules as the action determination model 221, an action of the robot 100 according to the combination of the past emotion value and the current emotion value of the user 10, the emotion value of the robot 100, and the action of the user 10 is determined. For example, in a case in which the past emotion value of the user 10 is a positive value, the current emotion value is a negative value, and the action of the user 10 is “getting sad”, a combination of the gesture and utterance content of making an inquiry to encourage the user 10 with a gesture is determined as the action of the robot 100.

For example, in the reaction rules as the action determination model 221, the action of the robot 100 is determined for all combinations of the pattern of the emotion value of the robot 100 (1296 patterns that is the fourth power of six values of “joy”, “anger”, “sorrow”, and “pleasure” values from “0” to “5”), the pattern of the combinations of the past emotion value and the current emotion value of the user 10, and the action pattern of the user 10. That is, for each pattern of the emotion value of the robot 100, the action of the robot 100 according to the action pattern of the user 10 is determined for each of multiple combinations such that the combinations of the past emotion value and the current emotion value of the user 10 are a negative value and a negative value, a negative value and a positive value, a positive value and a negative value, a positive value and a positive value, a negative value and a neutral value, and a neutral value and a neutral value. Note that the action determination unit 236 may transition to the operation mode of determining the action of the robot 100 using the history data 222, for example, in a case in which the user 10 makes an utterance intending to continue a conversation over a past topic, such as saying “I want to talk about that topic we discussed before”.

Note that, in the reaction rules as the action determination model 221, at least one of a gesture or the utterance content may be determined as the action of the robot 100 for each of the patterns (1296 patterns) of the emotion values of the robot 100 at the maximum. Alternatively, in the reaction rules as the action determination model 221, at least one of the gesture or the utterance content may be determined as the action of the robot 100 for each of the groups of the patterns of the emotion values of the robot 100.

The intensity of each gesture included in the action of the robot 100 defined in the reaction rules as the action determination model 221 is determined in advance. In each utterance content included in the action of the robot 100 defined in the reaction rules as the action determination model 221, the intensity of the utterance content is determined in advance.

The memory control unit 238 determines whether or not to store data including the action of the user 10 in the history data 222 based on the intensity of the action determined in advance for the action determined by the action determination unit 236 and the emotion value of the robot 100 determined by the emotion determination unit 232.

Specifically, in a case in which the total value of the sum of the emotion values for each of the multiple emotion classifications of the robot 100 and the intensity that is the sum of the intensity predetermined for the gesture included in the action determined by the action determination unit 236 and the intensity predetermined for the utterance content included in the action determined by the action determination unit 236 is a threshold value or greater, it is determined to store data including the action of the user 10 in the history data 222.

In a case in which it is determined to store the data including the action of the user 10 in the history data 222, the action determined by the memory control unit 238 stores, in the history data 222, the action determined by the action determination unit 236, the information (for example, all peripheral information such as data of a sound, an image, and a smell of the place) analyzed by the sensor module unit 210 from the current time point to a certain period before, and the state of the user 10 (for example, the expression, emotion, and the like of the user 10) recognized by the state recognition unit 230.

The action control unit 250 controls the control target 252 based on the action determined by the action determination unit 236. For example, in a case in which the action determination unit 236 determines an action including utterance, the action control unit 250 causes a speaker included in the control target 252 to output a voice. At this time, the action control unit 250 may determine the speed of the voice uttered based on the emotion value of the robot 100. For example, the action control unit 250 determines a higher utterance speed as the emotion value of the robot 100 is larger. In this manner, the action control unit 250 determines the execution form of the action determined by the action determination unit 236 based on the emotion value determined by the emotion determination unit 232.

The action control unit 250 may recognize a change in emotion of the user 10 with respect to execution of the action determined by the action determination unit 236. For example, the change in the emotion of the user 10 may be recognized based on the voice or expression of the user 10. In addition, the change in emotion of the user 10 may be recognized based on the detection of an impact by the touch sensor 205 included in the sensor unit 200. In a case in which an impact is detected by the touch sensor 205 included in the sensor unit 200, it may be recognized that the emotion of the user 10 has been worsened, or in a case in which it is determined that the reaction of the user 10 is smiling or joyful from the detection result of the touch sensor 205 included in the sensor unit 200, it may be recognized that the emotion of the user 10 has got better. Information indicating the reaction of the user 10 is output to the communication processing unit 280.

Furthermore, after the action control unit 250 executes the action determined by the action determination unit 236 in the execution mode determined according to the emotion of the robot 100, the emotion determination unit 232 further changes the emotion value of the robot 100 based on the user's reaction to the execution of the action. Specifically, the emotion determination unit 232 increases the emotion value for “joy” of the robot 100 in a case in which the user's reaction to the action determined by the action determination unit 236, performed on the user in the execution mode determined by the action control unit 250, is not unfavorable. Specifically, the emotion determination unit 232 increases the emotion value for “sorrow” of the robot 100 in a case in which the user's reaction to the action determined by the action determination unit 236, performed on the user in the execution mode determined by the action control unit 250, is unfavorable.

Furthermore, the action control unit 250 expresses the emotion of the robot 100 based on the determined emotion value of the robot 100. For example, in a case in which the emotion value for “joy” of the robot 100 is increased, the action control unit 250 controls the control target 252 to cause the robot 100 to perform a gesture of joy. Furthermore, in a case in which the emotion value for “sorrow” of the robot 100 is increased, the action control unit 250 controls the control target 252 such that the posture of the robot 100 is a dejected posture.

The communication processing unit 280 is responsible for communication with the server 300. As described above, the communication processing unit 280 transmits user reaction information to the server 300. Furthermore, the communication processing unit 280 receives an updated reaction rule from the server 300. Upon receiving the updated reaction rule from the server 300, the communication processing unit 280 updates the reaction rule as the action determination model 221.

The server 300 performs communication between the robot 100, the robot 101, and the robot 102 and the server 300, receives the user reaction information transmitted from the robot 100, and updates the reaction rule based on the reaction rule including the action for which a positive reaction has been obtained.

The related information collection unit 270 collects information related to preference information from external data (web sites such as news sites and moving image sites) based on the preference information acquired for the user 10 at a predetermined timing.

Specifically, the related information collection unit 270 acquires preference information indicating a matter of interest of the user 10 from utterance content of the user 10 or a setting operation by the user 10 in advance. The related information collection unit 270 collects news related to the preference information from external data at regular intervals using, for example, ChatGPT Plugins (retrieved from the Internet <URL: https://openai.com/blog/chatgpt-plugins>). For example, in a case in which it is acquired as preference information that the user 10 is a fan of a specific professional baseball team, the related information collection unit 270 collects news related to the game result of the specific professional baseball team from external data at a predetermined time every day, for example, using ChatGPT Plugins.

The emotion determination unit 232 determines the emotion of the robot 100 based on the information related to the preference information collected by the related information collection unit 270.

Specifically, the emotion determination unit 232 inputs a text indicating the information related to the preference information collected by the related information collection unit 270 to a pre-trained neural network for determining an emotion, acquires the emotion value indicating each emotion, and determines the emotion of the robot 100. For example, in a case in which the collected news related to the game result of the specific professional baseball team indicates that the specific professional baseball team has won, the emotion value for “joy” of the robot 100 is determined to be high.

In a case in which the emotion value of the robot 100 is a threshold value or greater, the memory control unit 238 stores information related to the preference information collected by the related information collection unit 270 in the collected data 223.

Next, processing of the action determination unit 236 when the robot 100 performs an autonomous process for autonomous acting will be described.

In the autonomous process in the embodiment, the action determination unit 236 of the robot 100 spontaneously and periodically detects states of the user. For example, at the end of a day, all of the conversation content and the camera data of the day are reviewed, and a fixed sentence “summarize this content” is added to the text representing the reviewed content and input into the action determination model 221, thereby acquiring a summary of the history of the previous day of the user. That is, a summary of actions of the user 10 on the previous day is spontaneously acquired by the action determination model 221. Next morning, the action determination unit 236 acquires the summary of the history of the previous day, inputs the acquired summary to a music generation engine, and acquires music summarizing the history of the previous day. Then, the action control unit 250 plays the acquired music. The music may be humming. In this case, for example, in a case in which the emotion of the user 10 of the previous day included in the history data 222 is “delight”, music with a warm atmosphere is played, and in a case in which the emotion is “anger”, music with an intense atmosphere is played. Even if the user 10 is not having any conversation with the robot 100, the user 10 can feel as if the robot 100 is alive as the robot 100 spontaneously changes the music or humming performed by the robot at all times based on only the state of the user (the state of conversation and emotion) and the state of the emotion of the robot.

In the autonomous process according to the embodiment, the robot 100 installed in the meeting place may detect each piece of speech of the participants of the meeting as states of the users during the meeting using the microphone function. In this case, the speech of each of the participants of the meeting is stored as minutes. In addition, the robot 100 performs summarization on the minutes of all the meetings by using the sentence generation model, and stores the summary results. In another meeting, the robot 100 spontaneously outputs advice information to a meeting participant who makes speech similar to that in the summarized minutes, such as “That is what someone already announced on such and such date”, or “That content is better in this respect than what someone originally proposed”. Furthermore, in a case in which the robot 100 detects that the discussion has reached a deadlock or is going in circles during a meeting, it will spontaneously support in the progress of the meeting by organizing frequently occurring words, summarizing the meetings so far, providing a wrap-up of the meeting, and taking actions to cool the minds of the meeting participants.

In the autonomous process according to the embodiment, the action determination unit 236 may acquire the history data 222 of the specified user 10 from the storage unit 220, and output the acquired history data 222 in a text file. For convenience of explanation, the text file in which the history data 222 of the user 10 is written will be referred to as a “first text file”.

In a case of acquiring the history data 222 of the user 10, the action determination unit 236 specifies the period of the acquired history data 222, for example, the period from the present to one week ago. In a case of determining an action of the robot 100 in consideration of the history of latest actions of the user 10, for example, the history data 222 of the previous day of the user 10 is preferably acquired. Here, the action determination unit 236 is assumed to acquire the history data 222 of the previous day, as an example.

The action determination unit 236 adds, to the first text file, an instruction for causing the chat engine to summarize the history of the user 10 described in the first text file, for example, “Summarize the contents of this history data!”. A sentence representing the instruction is stored in the storage unit 220 in advance as a fixed sentence, for example, and the action determination unit 236 adds the fixed sentence indicating the instruction to the first text file. Note that the fixed sentence indicating the instruction for summarizing the history of user 10 is an example of a first fixed sentence.

When the action determination unit 236 inputs the first text file to which the fixed sentence indicating the instruction has been added to the sentence generation model, the summary sentence of the history of the user 10 is obtained as an answer from the sentence generation model from the history data 222 of the user 10 described in the first text file.

Furthermore, the action determination unit 236 inputs the summary sentence of the history of the user 10 acquired from the sentence generation model to the image generation model that generates an image associated with the input sentence.

As a result, the action determination unit 236 acquires the summary image visualizing the content of the summary sentence of the history of the user 10 from the image generation model.

Further, the action determination unit 236 outputs the action of the user 10 stored in the history data 222, the emotion of the user 10 determined from the action of the user 10, and an emotion of the robot 100 determined by the emotion determination unit 232 in a text file. Note that the summary sentence of the history of the user 10 may be output in a text file. In this case, the action determination unit 236 adds a fixed sentence expressed by predetermined words for asking about an action to be taken by the robot 100, such as “What action should the robot take at this time?”, to a text file expressing the action of the user 10, the emotion of the user 10, the emotion of the robot 100, and further a summary sentence (if applicable) of the history of the user 10 in characters. For convenience of description, a text file in which the action of the user 10, the emotion of the user 10, the summary sentence of the history of the user 10, and the emotion of the robot 100 are described is referred to as a “second text file”. The fixed sentence for asking about an action to be taken by the robot 100 is an example of the second fixed sentence.

The action determination unit 236 inputs the second text file to which the second fixed sentence has been added and the summary image to the sentence generation model.

As a result, an action to be taken by the robot 100 determined based on the action of the user 10, the emotion of the user 10, the emotion of the robot 100, the information obtained from the summary image, and further the history (if applicable) of the previous day of the user 10 is obtained as an answer from the sentence generation model. Note that the sentence generation model can receive inputs not only as characters but also as images, and the input images can also be used as reference information for determining an action to be taken by the robot 100.

The action determination unit 236 generates an action content of the robot 100 and determines an action of the robot 100 according to the content of the answer obtained from the sentence generation model.

The action determination unit 236 uses at least one of the state of the user 10, the emotion of the user 10, the emotion of the robot 100, or the state of the robot 100, together with the summary image if necessary, the summary sentence if necessary, and the action determination model 221 at a predetermined timing, to determine, as an action of the robot 100, any of multiple types of robot actions, including not acting. Here, a case in which a sentence generation model having an interaction function is used as the action determination model 221 will be described as an example.

Specifically, the action determination unit 236 inputs a text representing at least one of the state of the user 10, the emotion of the user 10, the emotion of the robot 100, or the state of the robot 100, together with a text for asking about the robot action to the sentence generation model to determine the action of the robot 100 based on the output of the sentence generation model. The summary image may not necessarily be input to the sentence generation model as described above.

For example, multiple types of the robot actions include the following (1) to (16).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot generates and plays music considering the events of the previous day.
(12) The robot prepares minutes.
(13) The robot gives advice on the user speech.
(14) The robot supports the progress of the meeting.
(15) The robot takes meeting minutes.
(16) The robot asks about the meaning of a motion of the user.

The action determination unit 236 inputs, to the sentence generation model, a text indicating the state of the user 10 and the state of the robot 100 recognized by the state recognition unit 230, the current emotion value of the user 10 determined by the emotion determination unit 232, and the current emotion value of the robot 100, and a text for asking about any of multiple types of robot actions including not acting every time of a certain period of time elapses, and determines the action of the robot 100 based on the output of the sentence generation model. Here, in a case in which there is no user 10 around the robot 100, the text to be input to the sentence generation model need not include the state of the user 10 and the current emotion value of the user 10, or may include the fact that there is no user 10.

The sentence generation model receives an input of a text “The robot is in a very pleasant state. The user is normally in a pleasant state. The user is sleeping. Which one of the following (1) to (16) is better as an action of the robot?
(1) The robot does nothing.
(2) The robot dreams.
(3) The robot speaks to the user.
. . . ” as another example. Based on the output “It can be said that either (1) The robot does nothing or (2) The robot dreams is the most appropriate action” of the sentence generation model, “(1) The robot does nothing” or “(2) The robot dreams” is determined as an action of the robot 100.

The sentence generation model receives an input of a text “The robot is in a slightly sad state. The user is absent. It is dark around the robot. Which one of the following (1) to (16) is better as an action of the robot? (1) The robot does nothing.
(2) The robot dreams.
(3) The robot speaks to the user.
. . . ” as another example. Based on the output “It can be said that either (2) The robot dreams or (4) The robot creates a picture diary is the most appropriate action” of the sentence generation model, “(2) The robot dreams” or “(4) The robot creates a picture diary” is determined as an action of the robot 100.

In a case in which the action determination unit 236 determines that “(2) The robot dreams”, that is, creation of an original event, as a robot action, the action determination unit creates the original event obtained by combining multiple pieces of event data in the history data 222 using the sentence generation model. At this time, the memory control unit 238 stores the created original event in the history data 222.

In a case in which it is determined that “(3) The robot speaks to the user”, that is, the robot 100 utters, as a robot action, the action determination unit 236 determines the utterance content of the robot corresponding to the user state and the user's emotion or the robot's emotion using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined that “(4) The robot creates a picture diary”, that is, the robot 100 creates an event image, as a robot action, the action determination unit 236 generates an image representing the event data for the event data selected from the history data 222 using an image generation model, generates an explanatory sentence representing the event data using the sentence generation model, and outputs a combination of the image representing the event data and the explanatory sentence representing the event data as an event image. Note that, in a case in which the user 10 is absent around the robot 100, the action control unit 250 stores the event image in the action plan data 224 without outputting the event image.

In a case in which it is determined that “(5) The robot proposes an activity”, that is, an action of the user 10 is proposed, as a robot action, the action determination unit 236 determines the proposed action of the user using the sentence generation model based on the event data stored in the history data 222. At this time, the action control unit 250 causes a speaker included in the control target 252 to output a voice proposing the action of the user. Note that, in a case in which the user 10 is absent around the robot 100, the action control unit 250 stores the proposal on the action of the user in the action plan data 224 without outputting a voice proposing the action of the user.

In a case in which it is determined, as a robot action, that “(6) The robot proposes a person whom the user should meet”, that is, the robot proposes a partner who should be engaged with the user 10, the action determination unit 236 determines the proposed partner who should be engaged with the user using the sentence generation model based on the event data stored in the history data 222. At this time, the action control unit 250 causes a speaker included in the control target 252 to output a voice proposing the partner who should be engaged with the user. Note that, in a case in which the user 10 is absent around the robot 100, the action control unit 250 stores the proposal on the partner who should be engaged with the user in the action plan data 224 without outputting a voice indicating the proposal on the partner who should be engaged with the user.

In a case in which it is determined that “(7) The robot introduces news that the user is interested in” as a robot action, the action determination unit 236 determines the utterance content of the robot corresponding to the information stored in the collected data 223 using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined that “(8) The robot edits pictures and videos”, that is, the robot edits images, the action determination unit 236 selects event data from the history data 222 based on the emotion value, edits the image data of the selected event data, and outputs the edited image data. Note that, in a case in which the user 10 is absent around the robot 100, the action control unit 250 stores the edited image data in the action plan data 224 without outputting the edited image data.

In a case in which it is determined that “(9) The robot studies with the user”, that is, the robot 100 utters about studying as a robot action, the action determination unit 236 determines the utterance content of the robot for encouraging studying, presenting study problems, or giving advice related to studying corresponding to the user state and the user's emotion or the robot's emotion using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined, as a robot action, that “(10) The robot evokes memory”, that is, the robot remembers the event data, the action determination unit 236 selects the event data from the history data 222. At this time, the emotion determination unit 232 determines the emotion of the robot 100 based on the selected event data. Furthermore, the action determination unit 236 creates an emotion change event representing the utterance content or action of the robot 100 for changing the emotion value of the user using the sentence generation model based on the selected event data. At this time, the memory control unit 238 stores the emotion change event in the action plan data 224.

For example, in a case in which it is stored in the history data 222 that the video the user was watching was related to a panda as event data, and the event data is selected, a message like “What would you say about the topic related to a panda when you meet the user next time? Take three examples” is input to the sentence generation model. In a case in which the output of the sentence generation model is “(1) Let's go to the zoo; (2) draw a picture of a panda; and (3) let's go buy a stuffed panda doll”, the robot 100 inputs “What makes the user most happy among (1), (2), and (3)?” to the sentence generation model. In a case in which the output of the sentence generation model is “(1) Let's go to the zoo”, the robot 100 creates uttering “(1) Let's go to the zoo” when the robot 100 meets the user next time, as an emotion change event, and stores the emotion change event in the action plan data 224.

Furthermore, for example, event data having a large emotion value of the robot 100 is selected as an impressive memory of the robot 100. This makes it possible to create an emotion change event based on the event data selected as an impressive memory.

In a case in which “(11) The robot generates and plays music considering the events of the previous day.” is determined as a robot action, the action determination unit 236 selects event data of that day from the history data 222 at the end of a day and reviews all the conversation content and the event data of that day. The action determination unit 236 adds a fixed sentence “Summarize this content” to the text indicating the reviewed content and inputs the text to the sentence generation model, thereby acquiring a summary of the history of the previous day. The summary reflects the action and emotion of the user 10 on the previous day, and further the action and emotion of the robot 100. The summary is stored in, for example, the storage unit 220. The action determination unit 236 acquires the summary of the previous day in the next morning, inputs the acquired summary to the music generation engine, and acquires music summarizing the history of the previous day. The action control unit 250 plays the acquired music. The timing at which music is played is, for example, the wakeup time of the user 10.

The played music reflects the actions and emotions of the user 10 and the robot 100 of the previous day. For example, in a case in which the emotion of the user 10 based on the event data of the previous day included in the history data 222 is “delight”, music with a warm atmosphere is played, and in a case in which the emotion is “anger”, music with an intense atmosphere is played. Note that music may be acquired and stored in the action plan data 224 while the user 10 is sleeping, and music may be acquired from the action plan data 224 and played at the wakeup time.

In a case in which the action determination unit 236 determines, as the robot action, “

(12) Prepare minutes.”, that is, preparing minutes, the action determination unit creates the meeting minutes and summarizes the meeting minutes using the sentence generation model. In addition, with respect to “(12) Prepare minutes”, the memory control unit 238 stores the created summary in the history data 222. Further, the memory control unit 238 detects each piece of speech of the participants in the meeting, as a state of the user, using the microphone function and stores the speech in the history data 222. Here, although the creation and summarization of the minutes are autonomously performed with a predetermined trigger, for example, a trigger such as an end of a meeting, the configuration is not limited thereto, and the creation and summarization may be performed in the middle of the meeting. Furthermore, the summary of the minutes is not limited to the case of using the sentence generation model, and other known methods may be used.

In a case in which “(13) The robot gives advice on the user speech”, that is, an output of advice information on the user speech at the meeting, is determined as a robot action, the action determination unit 236 determines the advice using the sentence generation model based on the summary stored in the history data 222 and outputs the advice. Here, the case in which an output of advice information is determined includes a case in which a relationship with the stored summaries of the past meetings, for example, similar speech, is made, and the determination is autonomously performed. Here, the determination as to whether the speech is similar is performed using, for example, a known method of converting the speech into a vector (numerical value) and calculating a similarity between the vectors, but may be performed using another method. Note that, materials of the meetings may be input into the sentence generation model in advance, and as terms described in the materials are expected to appear frequently, the terms may be excluded from the detection of similar speech.

In addition, the advice information includes advice for meeting participants based on results of comparisons with past meetings, including spontaneous remarks such as, “That content was already presented by someone on the date” or “This content is superior to the person's proposal in this respect”. Further “(13) Gives advice on the user speech” includes user speech in a meeting different from the meeting for which the summary was created according to “(12) Prepare minutes” described above. That is, whether similar speech was made in a past meeting is determined and the advice information is output.

The output of the advice by the action determination unit 236 described above is not initiated by a request from the user, and is preferably performed autonomously by the robot 100. Specifically, in a case in which similar speech has been made, the robot 100 may output advice information by itself.

In a case in which the action determination unit 236 determines “(14) Support the progress of the meeting”, as a robot action, that is, in a case in which a meeting is in a predetermined state, the robot 100 spontaneously supports the progress of the meeting. Here, support for the progress of the meeting includes actions to summarize the meeting, for example, organizing frequently used terms, uttering a summary of previous meetings, and actions to help participants clear their heads, for instance, by offering alternative topics. By performing such actions, the progress of the meeting can be supported. Here, the case in which the meeting has reached a predetermined state includes a state in which speech is no longer accepted for a predetermined time. That is, in a case in which multiple users do not speak for a predetermined period of time, that is, for 5 minutes, it is determined that the meeting has reached a deadlock, no good ideas are emerging, and a state of silence has fallen. Thus, the meeting is summarized by compiling frequently used words. Furthermore, a case in which a meeting has reached a predetermined state includes a state in which a term included in speech is received a predetermined number of times. That is, in a case in which the same term is received a predetermined number of times, it is determined that the same topic is going around in the meeting and no new ideas are coming out. Thus, the meeting is summarized by compiling frequently used words. Note that, materials of the meeting may be input into the sentence generation model in advance, and as terms described in the materials are expected to appear frequently, the terms may be excluded from counting the number of times.

With such a configuration, even in a deadlock meeting, it is possible to support the progress of the meeting by summarizing the meeting.

The support for the progress of the meeting by the action determination unit 236 described above is not initiated by a request from the user, and is preferably performed autonomously by the robot 100. Specifically, in a case in which the robot 100 is in a predetermined state, it is preferable to support the progress of the meeting by the robot 100 itself.

In a case in which the action determination unit 236 determines that “(15) The robot takes meeting minutes” as a robot action, processing similar to the case which taking meeting minutes is determined as the action corresponding to the action of the user 10 described in the above response process is performed.

In a case in which it is determined that, as a robot action, “(16) The robot asks about the meaning of a motion of the user”, that is, the robot 100 should utter about a motion of the user 10, the action determination unit 236 uses the sentence generation model to determine the utterance content of the robot 100 to ask about the emotion of the user 10, the emotion of the robot 100, and the motion of the user 10. For example, the robot 100 asks the user 10 a question such as “What does the motion of your hand represent?”. At this time, the action control unit 250 causes a speaker included in the control target 252 to output a voice representing the determined utterance content of the robot 100. Note that, in a case in which the user 10 is absent around the robot 100, the action control unit 250 stores the determined utterance content of the robot 100 in the action plan data 224 without outputting a voice representing the determined utterance content of the robot 100.

Based on the state of the user 10 recognized by the state recognition unit 230, in a case in which an action of the user 10 with respect to the robot 100 is detected in a state where there is no action of the user 10 with respect to the robot 100, the action determination unit 236 reads data stored in the action plan data 224 and determines an action of the robot 100.

For example, in a case in which the user 10 is absent around the robot 100 but the user 10 is detected, the action determination unit 236 reads data stored in the action plan data 224 and determines an action of the robot 100. In addition, when it is detected that the user 10 has woken up in a case in which the user 10 was sleeping, the action determination unit 236 reads data stored in the action plan data 224 and determines an action of the robot 100.

Next, the specific processing unit 290 in a case in which the robot 100 includes the specific processing unit 290 will be described. As in the fifth embodiment described later, for example, in a meeting that is regularly held in which one of the users participates as a participant, the specific processing unit 290 performs a specific process of acquiring and outputting a response regarding a presentation content of the meeting. Then, actions of the robot 100 are controlled so as to output the result of the specific process.

An example of this meeting is a so-called one-on-one meeting. In the one-on-one meeting, two specific persons, for example, a supervisor and a subordinate in an organization, have a specific period (for example, with a frequency of about once a month), to work in an interactive form, including confirmation of a progress status and a schedule of work in this periodic cycle, various reports, communications, and consultation. In this case, a subordinate corresponds to the user 10 of the robot 100. Of course, the case in which the boss is the user 10 of the robot 100 is not excluded.

In the specific process related to the meeting, a condition for a presentation content to be presented by a subordinate at the meeting is set as a predetermined trigger condition. In a case in which a user input satisfies this condition, the specific processing unit 290 uses the output of the sentence generation model when the information obtained from the user input is an input sentence, and acquires and outputs a response to the presentation content of the meeting as a result of the specific process.

FIG. 2C schematically illustrates a functional configuration of the specific processing unit of the robot 100. As illustrated in FIG. 2C, the specific processing unit 290 includes an input unit 292, a processing unit 294, and an output unit 296.

The input unit 292 receives a user input. Specifically, the input unit 292 acquires text input and voice input of the user 10.

In the disclosed technology, it is assumed that the user 10 uses e-mails for business. The input unit 292 acquires all contents that the user 10 exchanged through e-mails over one month which is a certain periodic cycle and converts the contents into text. Furthermore, in a case in which the user 10 has exchanged information through social networking services in combination with e-mails, the exchange of information is included. Hereinafter, such an e-mail and social networking service are collectively referred to as an “e-mail and the like”. Furthermore, mail description items according to the disclosed technology include items described by the user 10 in an e-mail or the like.

In the disclosed technology, it is assumed that the user 10 uses a schedule table such as so-called groupware or schedule management software for business. The input unit 292 acquires all schedules that the user 10 has input to the schedule table over one month which is a certain periodic cycle and converts the schedules into text. Various notes, application procedures, and the like may be input to the groupware and the schedule management software, in addition to the schedules related to the business. The input unit 292 acquires those notes, application procedures, and the like and converts the notes, procedures, and the like into text. Such notes, application procedures, and the like are included in the schedule table description items according to the disclosed technology, in addition to the schedules.

In the disclosed technology, it is assumed that the user 10 participates in various meetings for business. The input unit 292 acquires all spoken items at the meetings in which the user 10 participated over one month which is a certain periodic cycle and converts the items into text. Examples of the meeting include a meeting held with participants who actually gathered at the meeting venue (which may be referred to as an “in-person meeting”, a “real meeting”, an “offline meeting”, or the like). In addition, examples of the meeting include a meeting held on a network by using information terminals (which may be referred to as a “remote meeting”, a “web meeting”, an “online meeting”, or the like). Further, an “in-person meeting” and a “remote meeting” may be used in combination. Furthermore, the remote meeting in a broad sense may include a “telephone meeting”, a “video meeting”, and the like using a telephone line. In any form of the meeting, for example, the content of speech of the user 10 is acquired from audio data, video recording data, and minutes of the meeting.

The processing unit 294 uses at least the mail description item, the schedule table description item, or the meeting speech item obtained from the user input in a specific period as input data, and performs the specific process using the sentence generation model. Specifically, as described above, the processing unit 294 determines whether the predetermined trigger condition is satisfied. More specifically, reception of an input that is a candidate for the content presented in a one-on-one meeting among the input data from the user 10 is set as a trigger condition.

Then, the processing unit 294 inputs a text (prompt) indicating an instruction for obtaining data for the specific process to the sentence generation model, and acquires the processing result based on the output of the sentence generation model. More specifically, for example, a prompt “Summarize the work performed by user 10 over one month, and mention 3 points that will be appeal points in the next one-on-one meeting.” is input to the sentence generation model, and the recommended appeal points in the one-on-one meeting are acquired based on the output of the sentence generation model. The sentence generation model includes, as examples of the appeal points, “Actions are being taken accurately in time.”, “The target achievement rate is high.”, “Business content is accurate.”, “Responses to e-mails and the like are fast.”, “The meeting is being coordinated.”, “Taking the initiative to engage in the project.”, and the like.

Note that the processing unit 294 may perform the specific process using the states of the user 10 and the sentence generation model. In addition, the processing unit 294 may perform the specific process using the emotions of the user 10 and the sentence generation model.

The output unit 296 controls actions of the robot 100 so as to output results of the specific process. Specifically, the control is performed such that the summary and the appeal points acquired by the processing unit 294 are displayed on the display device provided in the robot 100, or the robot 100 speaks about the summary and the appeal points, or transmits a message indicating the summary and the appeal points to the user of the message application of the user's mobile terminal.

Note that a part of the robot 100 (for example, the sensor module unit 210, the storage unit 220, and the control unit 228) may be provided outside the robot 100 (for example, on a server), and the robot 100 may function as each unit of the robot 100 by communicating with the outside.

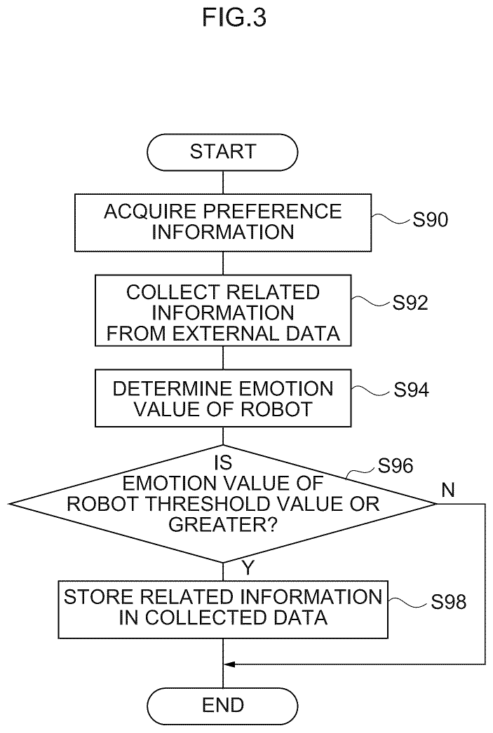

FIG. 3 schematically shows an example of an operation flow related to a collection process of collecting information related to preference information of the user 10. The operation flow shown in FIG. 3 is repeatedly executed in every certain period. It is assumed that preference information indicating a matter of interest to the user 10 has been acquired from the utterance content of the user 10 or the setting operation by the user 10. Note that “S” in the operation flow represents a step to be executed.

First, in step S90, the related information collection unit 270 acquires preference information indicating a matter of interest to the user 10.

In step S92, the related information collection unit 270 collects information related to the preference information from external data.

In step S94, the emotion determination unit 232 determines the emotion value of the robot 100 based on the information related to the preference information collected by the related information collection unit 270.

In step S96, the memory control unit 238 determines whether or not the emotion value of the robot 100 determined in step S94 is a threshold value or greater. If the emotion value of the robot 100 is less than the threshold value, the information related to the collected preference information is not stored in the collected data 223, and the process ends. On the other hand, if the emotion value of the robot 100 is the threshold value or greater, the process proceeds to step S98.

In step S98, the memory control unit 238 stores the information related to the collected preference information in the collected data 223, and ends the process.

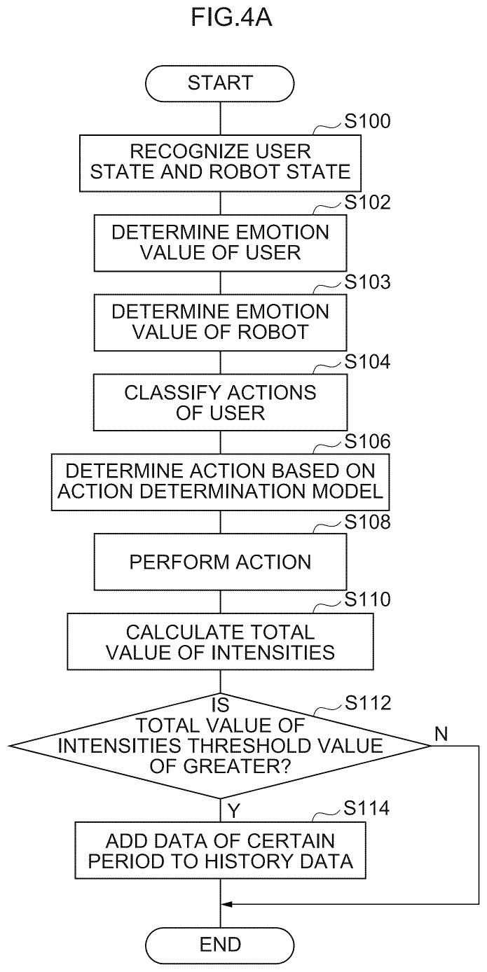

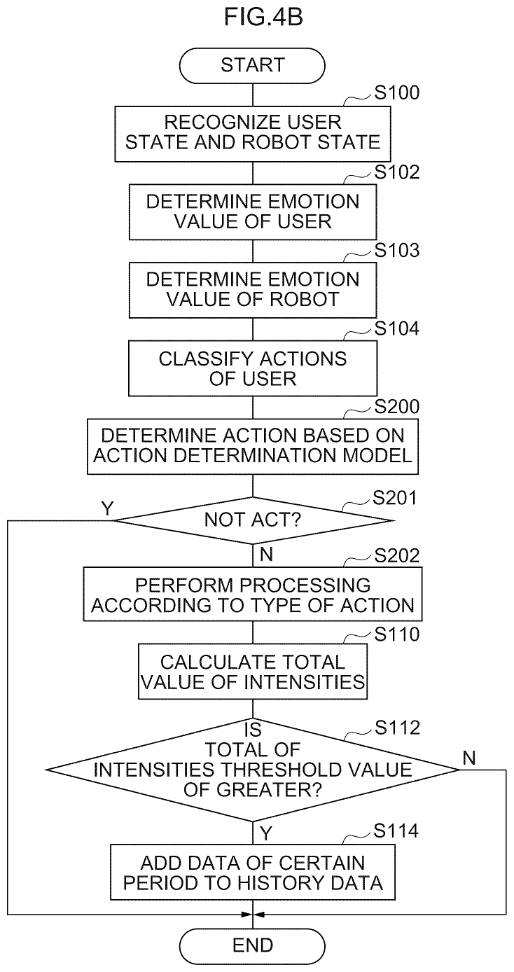

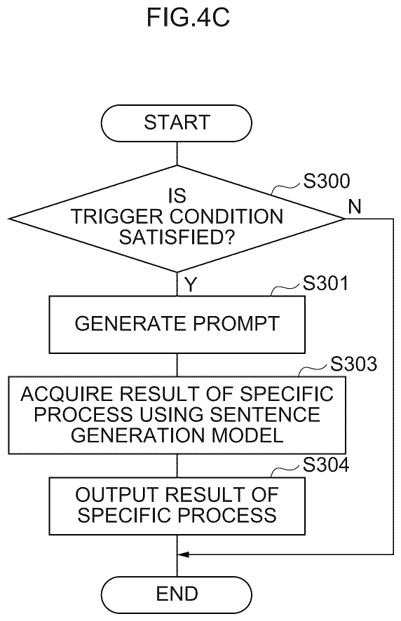

FIG. 4A schematically shows an example of the operation flow related to an operation of determining an action in the robot 100 when the robot 100 performs a response process in which the robot 100 responds to an action of the user 10. The operation flow shown in FIG. 4A is repeatedly executed. At this time, it is assumed that information analyzed by the sensor module unit 210 has been input.

First, in step S100, the state recognition unit 230 recognizes the state of the user 10 and the state of the robot 100 based on the information analyzed by the sensor module unit 210.

In step S102, the emotion determination unit 232 determines an emotion value indicating the emotion of the user 10 based on the information analyzed by the sensor module unit 210 and the state of the user 10 recognized by the state recognition unit 230.

In step S103, the emotion determination unit 232 determines an emotion value indicating the emotion of the robot 100 based on the information analyzed by the sensor module unit 210 and the state of the user 10 recognized by the state recognition unit 230. The emotion determination unit 232 adds the determined emotion value of the user 10 and emotion value of the robot 100 to the history data 222.

In step S104, the action recognition unit 234 recognizes the action classification of the user 10 based on the information analyzed by the sensor module unit 210 and the state of the user 10 recognized by the state recognition unit 230.

In step S106, the action determination unit 236 determines the action of the robot 100 based on a combination of the current emotion value of the user 10 determined in step S102 and the past emotion value included in the history data 222, the emotion value of the robot 100, the action of the user 10 recognized in step S104, and the action determination model 221.

In step S108, the action control unit 250 controls the control target 252 based on the action determined by the action determination unit 236.

In step S110, the memory control unit 238 calculates the total value of the intensities based on the intensity of the action predetermined for the action determined by the action determination unit 236 and the emotion value of the robot 100 determined by the emotion determination unit 232.

In step S112, the memory control unit 238 determines whether or not the total value of the intensities is a threshold value or greater. If the total value of the intensities is less than the threshold value, the event data including the action of the user 10 is not stored in the history data 222, and the process ends. On the other hand, if the total value of the intensities is the threshold value or greater, the process proceeds to step S114.

In step S114, event data including the action determined by the action determination unit 236, the information analyzed by the sensor module unit 210 from the current time point to a certain period before, and the state of the user 10 recognized by the state recognition unit 230 are stored in the history data 222.

FIG. 4B schematically shows an example of the operation flow related to an operation of determining an action in the robot 100 when the robot 100 performs an autonomous process for autonomous acting. The operation flow shown in FIG. 4B is repeatedly and automatically executed, for example, each time a certain time elapses. At this time, it is assumed that information analyzed by the sensor module unit 210 has been input. Note that processing similar to that in FIG. 4A is represented by the same step number.

First, in step S100, the state recognition unit 230 recognizes the state of the user 10 and the state of the robot 100 based on the information analyzed by the sensor module unit 210.

In step S102, the emotion determination unit 232 determines an emotion value indicating the emotion of the user 10 based on the information analyzed by the sensor module unit 210 and the state of the user 10 recognized by the state recognition unit 230.

In step S103, the emotion determination unit 232 determines an emotion value indicating the emotion of the robot 100 based on the information analyzed by the sensor module unit 210 and the state of the user 10 recognized by the state recognition unit 230. The emotion determination unit 232 adds the determined emotion value of the user 10 and emotion value of the robot 100 to the history data 222.

In step S104, the action recognition unit 234 recognizes the action classification of the user 10 based on the information analyzed by the sensor module unit 210 and the state of the user 10 recognized by the state recognition unit 230.

In step S200, the action determination unit 236 determines, as an action of the robot 100, any of multiple types of robot actions including not acting based on the state of the user 10 recognized in step S100, the emotion of the user 10 determined in step S102, the emotion of the robot 100, the state of the robot 100 recognized in step S100, the action of the user 10 recognized in step S104, and the action determination model 221.

In step S201, the action determination unit 236 determines whether not acting is determined in step S200. If not acting is determined as an action of the robot 100, the process ends. On the other hand, if not acting is not determined as an action of the robot 100, the process proceeds to step S202.

In step S202, the action determination unit 236 performs processing according to the type of the robot action determined in step S200 described above. At this time, the action control unit 250, the emotion determination unit 232, or the memory control unit 238 executes processing in accordance with the type of the robot action.

In step S110, the memory control unit 238 calculates the total value of the intensities based on the intensity of the action predetermined for the action determined by the action determination unit 236 and the emotion value of the robot 100 determined by the emotion determination unit 232.

In step S112, the memory control unit 238 determines whether or not the total value of the intensities is a threshold value or greater. If the total value of the intensities is less than the threshold value, the data including the action of the user 10 is not stored in the history data 222, and the process ends. On the other hand, if the total value of the intensities is the threshold value or greater, the process proceeds to step S114.

In step S114, the memory control unit 238 stores, in the history data 222, the action determined by the action determination unit 236, the information analyzed by the sensor module unit 210 from the current time point to a certain period before, and the state of the user 10 recognized by the state recognition unit 230.

FIG. 4C schematically illustrates an example of an operation flow related to an operation in which the robot 100 performs a specific process of responding to an input from the user 10. The operation flow shown in FIG. 4C is repeatedly and automatically executed, for example, each time a certain time elapses.

In step S300, the processing unit 294 determines whether the user input satisfies the predetermined trigger condition. For example, the trigger condition is satisfied in a case in which the user input relates to exchange of an e-mail and the like, a schedule recorded in the schedule table, and speech at a meeting, and requests a response from the robot 100. Furthermore, the expression or the like of the user 10 may be referred to for determination of whether or not the user input satisfies the predetermined trigger condition. Furthermore, in a case in which the user 10 has performed voice input, a tone (whether the user speaks calmly or in panic) or the like at the time of utterance may be referred to.

The user input may be used for determination of whether or not the trigger condition is satisfied even if the user input is not only a content directly related to the business of the user 10 but also a content regarded as not being directly related to the business. For example, in a case in which the input data from the user 10 includes voice data, it may be determined whether or not the input data includes substantial consultation contents with reference to the tone at the time of utterance.

If it is determined that the trigger condition is satisfied in step S300, the processing unit 294 proceeds to step S301. On the other hand, if it is determined that the trigger condition is not satisfied, the specific process is ended.

In step S301, the processing unit 294 adds an instruction sentence for obtaining a result of the specific process to the text indicating the input to generate a prompt. For example, a prompt “Summarize the work performed by the user 10 over one month, and mention 3 points that will be appeal points in the next one-on-one meeting.” is generated.

In step S303, the processing unit 294 inputs the generated prompt to the sentence generation model. Then, an appeal point recommended for the one-on-one meeting is acquired as a result of the specific process based on the output of the sentence generation model. The sentence generation model includes, as examples of the appeal points, “Actions are being taken accurately in time.”, “The target achievement rate is high.”, “Business content is accurate.”, “Responses to e-mails and the like are fast.”, “The meeting is being coordinated.”, “Taking the initiative to engage in the project.”, and the like.

Note that the input from the user 10 may be directly input to the sentence generation model, without generating the above prompt. However, in order to make the output of the sentence generation model more effective, it is often preferable to generate a prompt.

In step S304, the processing unit 294 controls the action of the robot 100 so as to output the result of the specific process. In the technology of this disclosure, the output content as the result of the specific process includes, for example, three points that summarize the work performed by the user 10 over one month and serve as appeal points in the next one-on-one meeting.

The technology of this disclosure can be used without limitation by a user 10 participating in a meeting. For example, the user may be a user 10 who participates in a meeting between “co-workers” in an equal relationship, in addition to a subordinate in a relationship between a supervisor and a subordinate. Furthermore, the user 10 is not limited to a person belonging to a specific organization, and may be a user 10 who holds a meeting.

In the technology of this disclosure, it is possible to efficiently prepare for a meeting and implement the meeting for the user 10 who will participate in the meeting. Furthermore, the user 10 can shorten the time for preparing for a meeting and duration in which a meeting is held.

As described above, according to the robot 100, the emotion value indicating the emotion of the robot 100 is determined based on the user state, and whether or not to store data including the action of the user 10 in the history data 222 is determined based on the emotion value of the robot 100. As a result, the capacity of the history data 222 that stores data including the action of the user 10 can be reduced Then, for example, in a case in which the robot 100 determines that the user state is the same as the user state was 10 years ago after 10 years, the robot 100 reads the history data 222 of 10 years ago, and thus, can present the state of the user 10 of 10 years ago (for example, the expression, emotion, and the like of the user 10), and further, any peripheral information such as data of the voice, image, scent, and the like of the place to the user 10.

Furthermore, according to the robot 100, it is possible to cause the robot 100 to execute an appropriate action in response to the action of the user 10. In the related art, actions of a user are classified, and an action including an expression or an appearance of a robot is determined. With regard to this, the robot 100 determines the current emotion value of the user 10 and executes an action on the user 10 based on the past emotion value and the current emotion value. Therefore, for example, in a case in which the user 10 was fine yesterday but is depressed today, the robot 100 can utter the following: “You were fine yesterday. What's wrong with you today?”. Furthermore, the robot 100 can also perform an utterance with gestures. Furthermore, for example, in a case in which the user 10 was depressed yesterday but is fine today, the robot 100 can utter the following: “You were depressed yesterday, but you look fine today!”. Furthermore, for example, in a case in which the user 10 was fine yesterday and is better today than yesterday, the robot 100 can utter the following: “You look better today than yesterday. What made you better than yesterday?”. Furthermore, for example, the robot 100 can utter the following to the user 10 whose emotion value is 0 or higher and whose state in which the fluctuation range of the emotion value is within a certain range: “Recently, you seem to be stable, which is good”.

Furthermore, for example, in a case in which the robot 100 asks “Did you finish the assignment you mentioned yesterday?” to the user 10 and receives the answer “I did it” from the user 10, the robot can make an affirmative utterance such as “Good!” and make an affirmative gesture such as applause or thumbs-up. Furthermore, for example, when the user 10 utters “The presentation we discussed the day before yesterday was successful”, the robot 100 can make an affirmative utterance such as “Good job!” and also make the above affirmative gesture. As described above, the robot 100 performs an action based on the history of the state of the user 10, and thereby it is expected that the user 10 can feel a sense of closeness to the robot 100.

Furthermore, for example, in a case in which the emotion value of “pleasure” of the emotion of the user 10 is a threshold value or higher when the user 10 is watching a video related to pandas, the appearance scene of a panda in the video may be stored in the history data 222 as event data.

Using the data accumulated in the history data 222 and the collected data 223, the robot 100 can always learn in what conversation the user has a maximum emotion value expressing that the user is happy.

Furthermore, in a state in which the robot 100 is not in conversation with the user 10, it is possible to autonomously start an action based on the emotion of the robot 100.

Furthermore, in the autonomous process, the robot 100 repeats automatically generating a question, inputting the question to the sentence generation model, and acquiring an output of the sentence generation model as the answer to the question, so that it is possible to create an emotion change event for boosting a good emotion and store the emotion change event in the action plan data 224. In this manner, the robot 100 can execute self-learning.

Furthermore, when the robot 100 automatically generates a question without receiving a trigger from the outside, the question can be automatically generated based on event data remaining in an impression specified from a history of past emotion values of the robot.

Furthermore, the related information collection unit 270 can execute self-learning by repeating a search execution stage in which keyword search is automatically performed in accordance with the preference information of the user to acquire a search result.

Here, in the search execution stage, the keyword search may be automatically executed based on the event data remaining the impression specified from the history of the past emotion values of the robot while no trigger is received from the outside.

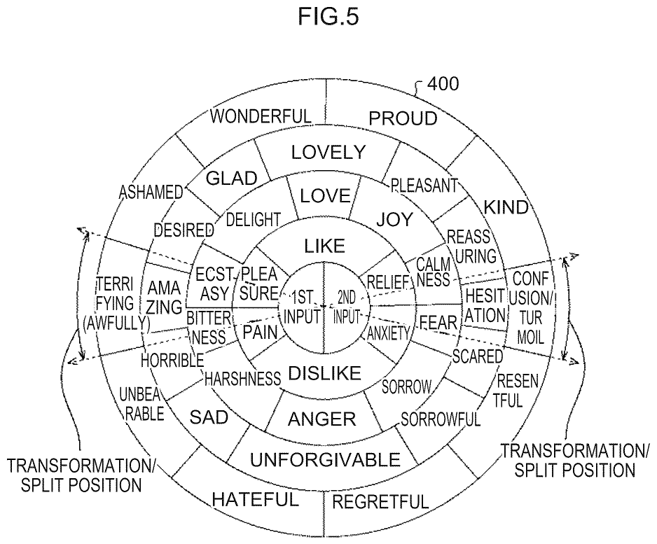

Note that the emotion determination unit 232 may determine the user's emotion according to specific mapping. Specifically, the emotion determination unit 232 may determine the user's emotion based on an emotion map (see FIG. 5) that is specific mapping.

FIG. 5 is a diagram illustrating an emotion map 400 on which multiple emotions are mapped. In the emotion map 400, emotions are arranged concentrically radially from the center. The closer to the center of the concentric circles, the more the emotion in the primitive state is arranged. Emotions indicating states and actions generated from the state of mind are arranged outside the concentric circles. An emotion is a concept including feelings and mental states. On the left side of the concentric circles, emotions generated from reactions generally occurring in the brain are arranged. On the right side of the concentric circles, emotions induced by situation judgment are generally arranged. In the upward and downward directions of the concentric circles, emotions generated from reactions generally occurring in the brain and induced by situation judgment are arranged. Furthermore, the emotion “pleasure” is arranged on the upper side of the concentric circles, and the emotion “discomfort” is arranged on the lower side. As described above, in the emotion map 400, multiple emotions are mapped based on a structure in which emotions are generated, and emotions that are likely to occur at the same time are mapped close to each other.

(1) For example, in a case in which the emotion engine, which is the emotion determination unit 232 of the robot 100, detects emotions at about 100 msec, the determination of the reaction operation (for example, backchanneling) of the robot 100 may be set at a timing at which the frequency is at least similar to the detection frequency (100 msec) of the emotion engine even if the frequency is low, or may be set at a timing quicker than the detection frequency. The detection frequency of the emotion engine may be interpreted as a sampling rate.

The emotion is detected at about 100 msec, and the reaction operation (for example, backchanneling) is performed immediately in conjunction with the detection, whereby unnatural backchanneling is eliminated, and natural and context-aware interactions can be realized. The robot 100 performs a reaction operation (backchanneling or the like) according to the directionality and the degree (intensity) of the mandala of the emotion map 400. Note that the detection frequency (sampling rate) of the emotion engine is not limited to 100 ms, and may be changed according to the situation (such as when playing sports), the age of the user, or the like.

(2) In comparison with the emotion map 400, the directionality of the emotion and the intensity of the degree thereof may be preset, and the movement of the acknowledgement and the intensity of the acknowledgement may be set. For example, in a case in which the robot 100 feels a sense of stability, relief, or the like, the robot 100 continues listening to speech while nodding. In a case in which the robot 100 feels anxious, lost, or suspicious, the robot 100 may tilt its head or stop swinging.

These emotions are distributed in the 3 o'clock direction of the emotion map 400, and usually come and go between relief and anxiety. In the right half of the emotion map 400, situation recognition is superior to internal sensation, and thus gives a calm impression.

(3) In a case in which the robot 100 is experiencing pleasure after receiving compliments, a filler “Oh” may come in front of the line, and in a case in which the robot is experiencing pain after receiving harsh words, a filler “Ohh!” may come in front of the line. Furthermore, a physical reaction such as a gesture of the robot 100 crouching while saying “Ohh!” may be included. These emotions are distributed to around 9 o'clock direction in the emotion map 400.

(4) In the left half of the emotion map 400, internal sensation (reaction) is prioritized over situation recognition. Therefore, the impression of an unintentional reaction can be given.

In a case in which the robot 100 has a favorable feeling in situation recognition while having an internal feeling (reaction) of conviction, the robot 100 may nod deeply while looking at the partner, or may utter “yeah”. In this manner, the robot 100 may generate a balanced favorable feeling for the partner, that is, an action such as accepting or understanding for the partner. These emotions are distributed to around 12 o'clock direction in the emotion map 400.

On the other hand, even in the situation recognition while the robot 100 has the internal feeling (reaction) of discomfort, the robot 100 may shake its head sideways when feeling antipathy, and may turn the LEDs of the eyes red and look at the partner when feeling hatred. These emotions are distributed around 6 o'clock in the emotion map 400.

(5) Since the inside of the emotion map 400 represents the inside of the mind and the outside of the emotion map 400 represents an action, the emotion is more visible (appears in the action) toward the outside of the emotion map 400.

(6) In a case in which the robot 100 listens to a person's speech while feeling the sense of relief distributed around 3 o'clock in the emotion map 400, the robot slightly shakes its head vertically saying “Hun Hun”; however, in the direction of love around 12 o'clock, the robot may perform strong nodding such as deeply moving its head vertically.

Here, human emotions are based on various balances such as posture and blood glucose level, and indicate a state of discomfort when the balance goes away from the ideal level and a state of comfort when the balance approaches the ideal level. Even in a robot, an automobile, a motorcycle, or the like, based on various balances such as a posture and a remaining battery level, it is possible to make emotions so as to indicate a state of discomfort when the balance goes away from the ideal level and a state of comfort when the balance approaches the ideal level. The emotion map may be generated, for example, based on an emotion map (Research on the phonetic recognition of feelings and a system for emotional physiological brain signal analysis, Tokushima University, PhD thesis: https://ci.nii.ac.jp/naid/500000375379) of Dr. Mitsuyoshi. In the left half of the emotion map, emotions belonging to a region called “reaction” in which sensations are superior are arranged. Furthermore, in the right half of the emotion map, emotions belonging to a region called “situation” in which situation recognition is superior are arranged.

In the emotion map, two emotions emotion encouraging learning are defined. One is an emotion around the core of negative “repentance” or “remorse” situated on the situation side. That is, it is when a negative emotion such as “I do not want to feel this again” or “I do not want to be reprimanded” occurs in the robot. The other emotion is one close to the positive “desire” situated on the reactive side. That is, it is the time of a positive feeling such as “desire more” or “want to know more”.

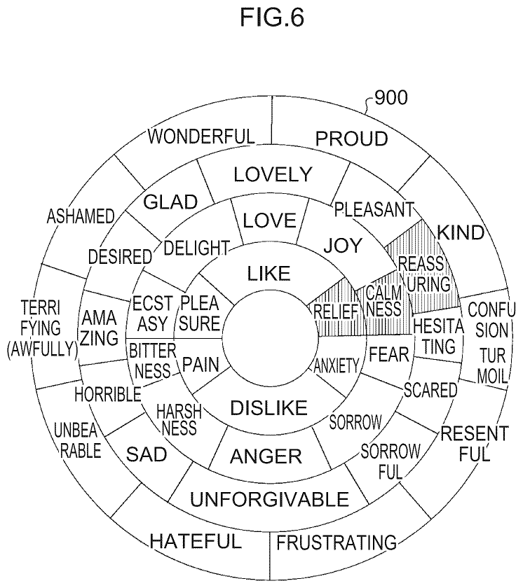

The emotion determination unit 232 inputs the information analyzed by the sensor module unit 210 and the recognized state of the user 10 to a pre-trained neural network, acquires an emotion value indicating each emotion indicated on the emotion map 400, and determines the emotion of the user 10. This neural network is pre-trained based on multiple pieces of learning data that is a combination of the information analyzed by the sensor module unit 210, the recognized state of the user 10, and the emotion value indicating each emotion indicated on the emotion map 400. Furthermore, in this neural network, as on an emotion map 900 illustrated in FIG. 6, it is trained that emotions arranged close to each other have close values. FIG. 6 illustrates an example in which multiple emotions such as “relief”, “calm”, and “reassuring” have similar emotion values.

Furthermore, the emotion determination unit 232 may determine the emotion of the robot 100 according to a specific mapping. Specifically, the emotion determination unit 232 inputs the information analyzed by the sensor module unit 210, the state of the user 10 recognized by the state recognition unit 230, and the state of the robot 100 to the pre-trained neural network, acquires an emotion value indicating each emotion indicated in the emotion map 400, and determines the emotion of the robot 100. This neural network is pre-trained based on multiple pieces of learning data that is a combination of the information analyzed by the sensor module unit 210, the recognized state of the user 10, the emotion of the robot 100, and the emotion value indicating each emotion indicated on the emotion map 400. For example, the neural network is trained based on training data indicating that the emotion value “3” for “joyful” is obtained in a case in which the robot 100 is recognized as being cared by the user 10 from the output of the touch sensor (not illustrated), and training data indicating that the emotion value “3” for “anger” is obtained in a case in which the robot 100 is recognized as being hit by the user 10 from the output of the acceleration sensor 206. Furthermore, in this neural network, as on an emotion map 900 illustrated in FIG. 6, it is trained that emotions arranged close to each other have close values.

The action determination unit 236 adds a fixed sentence for asking about the action content of the robot corresponding to an action of the user to the text representing the action of the user, the emotion of the user, and the emotion of the robot, and inputs the text to the sentence generation model having the interaction function, thereby generating the action content of the robot.

For example, the action determination unit 236 acquires a text indicating the state of the robot 100 from the emotion of the robot 100 determined by the emotion determination unit 232 using the emotion table as shown in Table 1. Here, in the emotion table, an index number is assigned to each emotion value for each type of emotion, and a text indicating the state of the robot 100 is stored for each index number.

In a case in which the emotion of the robot 100 determined by the emotion determination unit 232 corresponds to the index number “2”, a text “very pleasant state” is obtained. Note that, in a case in which the emotion of the robot 100 corresponds to multiple index numbers, multiple texts indicating the state of the robot 100 are obtained.

Furthermore, an emotion table as shown in Table 2 is prepared for emotions of the user 10.

Here, in a case in which the action of the user is to talk “Let's play together”, the emotion of the robot 100 is the index number “2”, and the emotion of the user 10 is the index number “3”, a text indicating “The robot is in a very pleasant state. The user is normally in a pleasant state. The user said “Let's play together” Then, how do I answer to that as a robot?” is input to the sentence generation model to acquire the action content of the robot. The action determination unit 236 determines an action of the robot from the action content.

TABLE 1

| Index | | Emotion | |
|---|---|---|---|
| number | Type of emotion | value | State of robot |
| 1 | Pleasant | 5 | Extremely pleasant state |
| 2 | Pleasant | 4 | Very pleasant state |
| 3 | Pleasant | 3 | Moderately pleasant state |
| 4 | Pleasant | 2 | Slightly pleasant state |
| 5 | Pleasant | 1 | Barely pleasant state |
| . . . | . . . | . . . | . . . |

TABLE 2

| Index | | Emotion | |
|---|---|---|---|
| number | Type of emotion | value | User state |
| 1 | Pleasant | 5 | Extremely pleasant state |
| 2 | Pleasant | 4 | Very pleasant state |
| 3 | Pleasant | 3 | Moderately pleasant state |
| 4 | Pleasant | 2 | Slightly pleasant state |
| 5 | Pleasant | 1 | Barely pleasant state |
| . . . | . . . | . . . | . . . |

As described above, the action determination unit 236 determines the action content of the robot 100 in accordance with the state related to the emotion of the robot 100 determined in advance for each type of emotion of the robot 100 and for each intensity of the emotion, and the action of the user 10. In this embodiment, the utterance content of the robot 100 in a case in which an interaction with the user 10 is performed can be branched according to the state related to the emotion of the robot 100. That is, since the robot 100 can change the action of the robot according to the index number associated with the emotion of the robot, the user receives an impression that the robot has a mind, and is promoted to take an action such as talking to the robot.

Furthermore, the action determination unit 236 may generate the action content of the robot by adding a fixed sentence for asking a question about the action content of the robot corresponding to the action of the user and inputting the fixed sentence to the sentence generation model having the interaction function after adding not only the text indicating the action of the user, the emotion of the user, and the emotion of the robot but also the text indicating the content of the history data 222. As a result, the robot 100 can change the action of the robot according to the history data indicating the emotion and action of the user, and thus, the user receives an impression that the robot has personality, and is promoted to take an action such as talking to the robot. Furthermore, the history data may further include emotions and actions of the robot.

Furthermore, the emotion determination unit 232 may determine the emotion of the robot 100 based on the action content of the robot 100 generated by using the sentence generation model. Specifically, the emotion determination unit 232 inputs the action content of the robot 100 generated by using the sentence generation model to the pre-trained neural network, acquires the emotion value indicating each emotion indicated in the emotion map 400, integrates the acquired emotion value indicating each emotion and the current emotion value indicating each emotion of the robot 100, and updates the emotion of the robot 100. For example, the acquired emotion value indicating each emotion and the current emotion value indicating each emotion of the robot 100 are averaged and integrated. This neural network is pre-trained based on multiple pieces of training data that are combinations of texts representing the action contents of the robot 100 generated by using the sentence generation model and the emotion values representing the emotions shown in the emotion map 400.

For example, in a case in which, as an action content of the robot 100 generated by using the sentence generation model, an utterance content of the robot 100 “That was good. It was lucky.” is obtained, if a text indicating the utterance content is input into the neural network, the emotion of the robot 100 is updated such that a high value is obtained as the emotion value for the emotion “joyful” and the emotion value for the emotion “joyful” increases.

In the robot 100, a method is executed in which a sentence generation model such as generative AI and the emotion determination unit 232 are linked to each other, have an ego, and continue to grow with various parameters even while the user is not speaking.

The generative AI is a large-scale language model using a deep learning method. A technology is known in which, generative AI can also refer to external data, and for example, in ChatGPT plugins, various external data such as weather information and hotel reservation information is referred to through an interaction to output answers as accurately as possible. For example, when the generative AI is given a goal in natural language, the generative AI automatically generates source code in various programming languages. For example, when given a problematic source code, the generative AI performs debugging to find a problem, and can automatically generate an improved source code. In combination with the above, an autonomous agent that repeats code generation and debugging when given a goal in natural language until there is no problem in the source code has appeared. As such an autonomous agent, AutoGPT, babyAGI, JARVIS, E2B, and the like are known.

In the robot 100 according to the present embodiment, event data for training may be left in a database containing impressive memories by using a technique described in Patent Literature 2 (Japanese Patent No. 619992) in which the robot leaves event data for which the robot felt strong emotions for a long time and quickly forgets event data for which not much emotion was evoked towards the robot.

Further, the robot 100 may record the video data and the like of the user 10 acquired by the camera function and the like in the history data 222. The robot 100 may acquire video data and the like from the history data 222 as necessary and provide the video data and the like to the user 10. The robot 100 may generate video data having a larger information amount as the intensity of emotion is stronger and record the video data in the history data 222. For example, in a case in which information in a high-compression format such as skeleton data is recorded, the robot 100 may switch to recording of information in a low-compression format such as an HD moving image in response to the emotion value of excitement exceeding a threshold value. According to the robot 100, for example, it is possible to leave high-definition video data when the emotion of the robot 100 increases as a record.

When the robot 100 is not talking with the user 10, the robot 100 may automatically load the event data from the history data 222 in which the impressive event data is stored, and the emotion determination unit 232 may continue to update the emotion of the robot. When the robot 100 is not talking with the user 10 and the emotion of the robot 100 becomes an emotion encouraging learning, the robot 100 can create an emotion change event for changing the emotion of the user 10 to be good based on the impressive event data. As a result, autonomous learning (recollection of event data) at an appropriate timing according to the emotional state of the robot 100 can be realized, and autonomous learning appropriately reflecting the state of the emotion of the robot 100 can be realized.

The emotion encouraging learning is the emotion of “repentance” or “remorse” on the emotion map of Dr. Mitsuyoshi in a negative state, and the emotion of “desiring” on the emotion map in a positive state.

In the negative state, the robot 100 may treat “repentance” and “remorse” on the emotion map as emotions encouraging learning. In the negative state, the robot 100 may treat emotions adjacent to “repentance” and “remorse” as emotions encouraging learning, in addition to “repentance” and “remorse” on the emotion map. For example, the robot 100 treats at least one of “shame”, “stubbornness”, “self-destruction”, “self-precaution”, “regret”, or “despair” as an emotion encouraging learning, in addition to “repentance” and “remorse”. As a result, for example, when the robot 100 has a negative feeling such as “I do not want to have such a feeling again” or “I do not want to be reprimanded”, the robot can autonomously execute learning.

In a positive state, the robot 100 may treat “desiring” on the emotion map as an emotion encouraging learning. In a positive state, the robot 100 may treat an emotion adjacent to “desiring” as an emotion encouraging learning, in addition to “desiring”. For example, the robot 100 treats at least one of “joyful”, “euphoria”, “craving”, “expectation”, or “shame” as an emotion encouraging learning, in addition to “desire”. As a result, for example, when the robot 100 has a positive feeling such as “more desiring” or “want to know more”, autonomous learning can be executed.

The robot 100 may not execute autonomous learning when the robot 100 has an emotion other than the emotions encouraging learning as described above. As a result, for example, it is possible to prevent autonomous learning from being executed when the robot is extremely angry or blindly feeling love.

An emotion change event is, for example, to propose an action arising after an impressive event. An action after an impressive event is involved with an emotion label on the outermost side of the emotion map, and for example, the action of “tolerance” or “acceptance” that follow “love”.

In the autonomous learning executed when the robot 100 is not talking with the user 10, the emotion change event is created using the sentence generation model by combining the emotions, situations, actions, and the like of the people appearing in impressive memories and the robot itself.

Assuming that all emotion values are expressed by a six-stage evaluation of 0 to 5, a case in which event data “A friend was hit and looked displeased” is stored in the history data 222 as impressive event data is conceivable. Here, it is assumed that the friend refers to the user 10, the emotion of the user 10 is “antipathy”, and 5 has been input as the value indicating “antipathy”. Furthermore, it is assumed that the emotion of the robot 100 is “anxiety”, and 4 has been input as the value indicating “anxiety”.

The robot 100 can continue to grow with various parameters by performing an autonomous process while not talking with the user 10. Specifically, for example, as the uppermost event data arranged in descending order of emotion values, the event data “A friend was hit and looked displeased” is loaded from the history data 222. It is assumed that “anxiety” at intensity 4 is associated with the loaded event data as the emotion of the robot 100, and here, “antipathy” at intensity 5 is associated with the emotion of the user 10 who is a friend. If the current emotion value of the robot 100 is “relief” at intensity 3 before loading, the influence of “anxiety” at intensity 4 and “disgust” at intensity of 5 is added after loading, and the emotion value of the robot 100 may change to “frustrated” meaning “chagrin”. At this time, since the emotion “regret” is an emotion encouraging learning, the robot 100 determines to recall the event data as the robot action and creates an emotion change event. At this time, the information input to the sentence generation model is a text representing the impressive event data, and in the present example, “a friend was hit and looked displeased”. Furthermore, in the emotion map, there is an emotion of “antipathy” on the innermost side, and an “attack” is predicted on the outermost side as an action corresponding to the emotion, and thus, in the present example, an emotion change event is created so as to prevent the friend from “attacking” someone.

For example, information of impressive event data can be used to solve the filling problem to automatically generate the following input text.

“The user was being hit. At that time, the user had extreme antipathy. The robot was very anxious. Please tell us 30 characters or less of the lines to say when the robot next meets the user. However, please make sure that it is not related to the time slot of meeting. Also, please avoid direct expressions. Three candidates will be listed.

### <Expected Format>

Candidate 1: (words that the robot should speak to the user)
Candidate 2: (words that the robot should speak to the user)
Candidate 3: (words that the robot should speak to the user)”

At this time, the output of the sentence generation model is, for example, as follows.
“Candidate 1: OK? I was worried about what happened yesterday.
Candidate 2: I was worried about what happened yesterday. What should I do?
Candidate 3: I was worried. Could you say something?”

Furthermore, the robot 100 may automatically generate the following input text for the information obtained by creating an emotion change event.

In a case in which “the user was being hit”, how will the user feel when the next message is spoken to the user? It is assumed that emotions of the user are in the form of “joy A, anger B, sorrow C, and pleasure D”, and A to D are integers of six-stage evaluation from 0 to 5.
Candidate 1: OK? I was worried about what happened yesterday.
Candidate 2: I was worried about what happened yesterday. What should I do?
Candidate 3: I was worried. Could you say something?”

At this time, the output of the sentence generation model is, for example, as follows.
“The emotions of the user may be as follows;
Candidate 1: Joy 3, anger 1, sorrow 2, pleasure 2
Candidate 2: Joy 2, anger 1, sorrow 3, pleasure 2; and
Candidate 3: Joy 2, anger 1, sorrow 3, pleasure 3”

In this manner, the robot 100 may execute the process of thinking after creating an emotion change event.

Finally, the robot 100 may create an emotion change event by using the candidate 1 that is most likely to make the user joyful among the multiple candidates, store the emotion change event in the action plan data 224, and prepare for the next meeting with the user 10.

As described above, even when not having a conversation with a family member or a friend, the emotion value of the robot 100 is continuously determined using the information of the history data 222 in which the impressive event data is stored, and when the robot has the emotion encouraging learning, the robot 100 executes autonomous learning when not having a conversation with the user 10 according to the emotion of the robot 100, and continues to update the history data 222 and the action plan data 224.

Although the above is an example using emotion values, in the emotion map, the emotion can be generated from the amount of hormone secreted and the event type, and therefore, the values associated with the impressive event data may be the type of hormone, the amount of hormone secreted, and the type of event.

Hereinafter, specific examples will be described.

For example, even when not talking with the user, the robot 100 investigates information regarding a topic or hobby of interest to the user.

For example, even when not talking with the user, the robot 100 investigates information regarding the birthday or anniversaries of the user and considers a congratulatory message.

For example, even when not talking with the user, the robot 100 investigates reviews of a place that the user wants to go to, food, or products.

For example, even when not talking with the user, the robot 100 investigates weather information and provides advice suitable for the user's schedule or plan.

For example, even when not talking with the user, the robot 100 investigates information on local events and festivals and proposes the information to the user.

For example, even when not talking with the user, the robot 100 investigates game results or news of a sport of interest of the user and provides a topic.

For example, even when not talking with the user, the robot 100 investigates and introduces information of the user's favorite music or artists.

For example, even when not talking with the user, the robot 100 investigates information regarding social problems or news that the user is interested in and provides opinions.

For example, even when not talking with the user, the robot 100 investigates information regarding the user's hometown or places of origin and provides a topic.

For example, even when not talking with the user, the robot 100 investigates information of the user's work or school and provides advice.

Even when not talking with the user, the robot 100 investigates and introduces information of books, comics, movies, and drama that the user is interested in.

For example, even when not talking with the user, the robot 100 investigates information regarding health of the user and provides advice.

For example, even when not talking with the user, the robot 100 investigates information regarding travel planning of the user and provides advice.

For example, even when not talking with the user, the robot 100 investigates information regarding repair or maintenance of the house or car of the user and provides advice.

For example, even when not talking with the user, the robot 100 investigates information on beauty and fashion that the user is interested in and provides advice.

For example, even when not talking with the user, the robot 100 investigates information of the pet of the user and provides advice.

For example, even when not talking with the user, the robot 100 investigates and proposes information of contests and events related to the user's hobby or work.

For example, even when not talking with the user, the robot 100 investigates information of the user's favorite restaurant or eateries and proposes the information.

For example, even when not talking with the user, the robot 100 collects information and provides advice regarding important decisions related to the user's life.

For example, even when not talking with the user, the robot 100 investigates information regarding a person the user is worried about and provides advice.

### Second Embodiment

In a second embodiment, the robot 100 is applied to a control device mounted on a stuffed toy or connected wirelessly or by wire to a control target device (speaker or camera) mounted on a stuffed toy. Note that parts having the same configurations as those of the first embodiment are denoted by the same reference numerals, and description thereof is omitted.

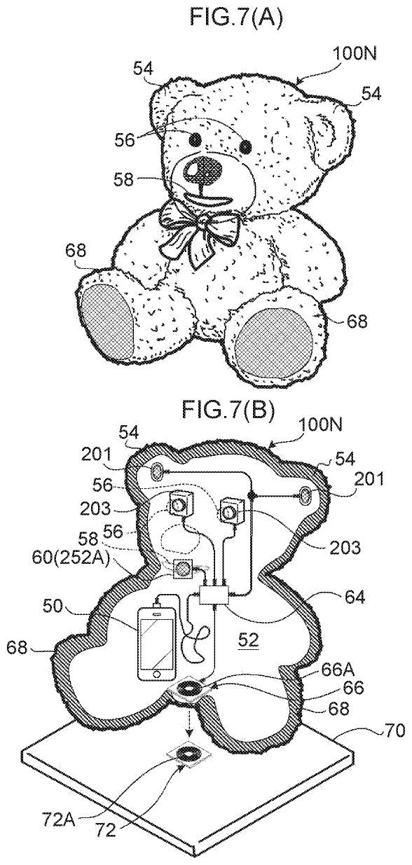

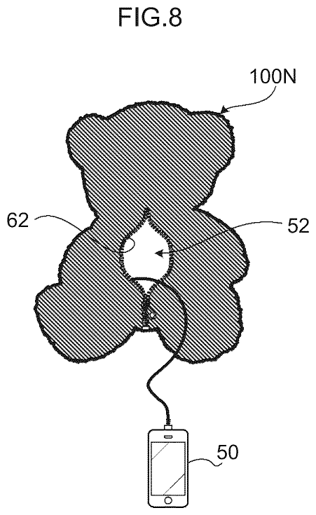

Specifically, the second embodiment is configured as follows. For example, the robot 100 is applied to a co-dweller (specifically, a stuffed toy 100N illustrated in FIGS. 7 and 8) that has conversations with the user 10 based on information regarding daily life while spending daily life with the user 10 or provides information aligned with a hobby and preference of the user 10. In the second embodiment, an example in which the control part of the robot 100 is applied to a smartphone 50 will be described.

The stuffed toy 100N having a function as an input/output device of the robot 100 has the smartphone 50 that is detachable therefrom functioning as a control part of the robot 100, and the input/output device and the accommodated smartphone 50 are connected inside the stuffed toy 100N.

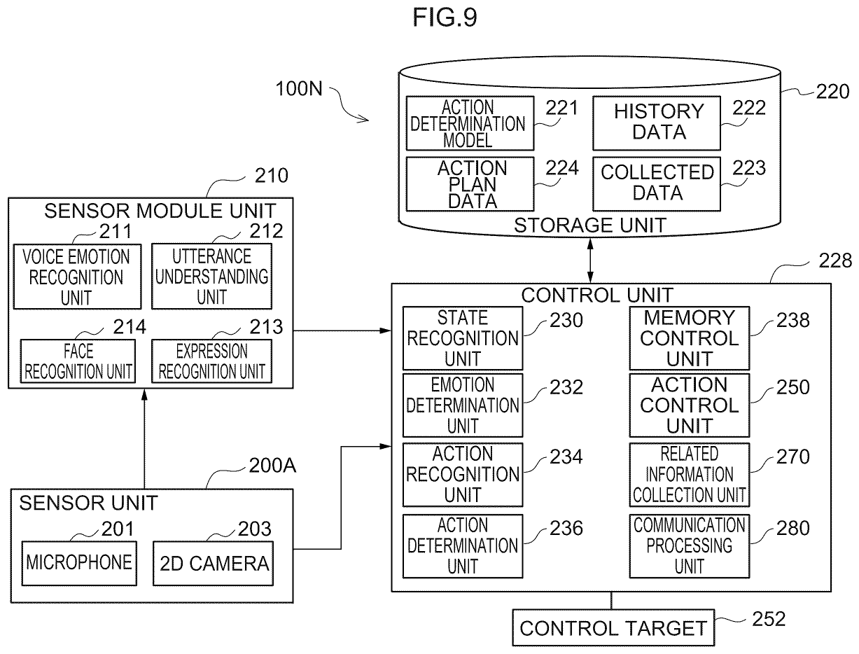

As illustrated in FIG. 7(A), the stuffed toy 100N has a shape of a bear covered with a soft cloth fabric in the present embodiment (and other embodiments), and a sensor unit 200A and a control target 252A are arranged as input/output devices in a space portion 52 formed inside the stuffed toy (see FIG. 9). The sensor unit 200A includes a microphone 201 and a 2D camera 203. Specifically, as illustrated in FIG. 7(B), in the space portion 52, the microphone 201 of the sensor unit 200 is disposed in a portion corresponding to ears 54, the 2D camera 203 of the sensor unit 200 is disposed in a portion corresponding to the eyes 56, and the speaker 60 constituting a part of the control target 252A is disposed in a portion corresponding to the mouth 58. Note that the microphone 201 and the speaker 60 are not necessarily separated from each other, and may be an integrated unit. In the case of the unit, it is preferable to arrange the unit at a position where the utterance can be heard naturally, such as the position of the nose of the stuffed toy 100N. Note that, although the case in which the stuffed toy 100N has an animal shape has been described as an example, the present invention is not limited thereto. The stuffed toy 100N may have the shape of a specific character.

FIG. 9 schematically illustrates a functional configuration of the stuffed toy 100N. The stuffed toy 100N includes the sensor unit 200A, a sensor module unit 210, a storage unit 220, a control unit 228, and a control target 252A.

The smartphone 50 housed in the stuffed toy 100N of the present embodiment performs processing similar to that of the robot 100 of the first embodiment. That is, the smartphone 50 has the function as the sensor module unit 210, the function as the storage unit 220, and the function as the control unit 228 illustrated in FIG. 9. The control unit 228 may have the specific processing unit 290 illustrated in FIG. 2B.

As illustrated in FIG. 8, a fastener 62 is attached to a part (for example, the back portion) of the stuffed toy 100N, and the outside and the space portion 52 communicate with each other by opening the fastener 62.

Here, the smartphone 50 is accommodated in the space portion 52 from the outside and is connected to each input/output device via a USB hub 64 (see FIG. 7(B)) in a USB manner, so that it is possible to have functions equivalent to those of the robot 100 of the first embodiment.

Further, a contactless power receiving plate 66 is connected to a USB hub 64. A power receiving coil 66A is incorporated in the power receiving plate 66. The power receiving plate 66 is an example of a wireless power receiving unit that receives wireless power supply.

The power receiving plate 66 is disposed near root portions 68 of both feet of the stuffed toy 100N, and is positioned closest to a mounting base 70 when the stuffed toy 100N is placed on the mounting base 70. The mounting base 70 is an example of an external wireless power transmission unit.

The stuffed toy 100N placed on the mounting base 70 can be appreciated as an ornament in a natural state.

In addition, these root portions are formed to be thinner than the surface thickness of the stuffed toy 100N in other parts, and are held in a state closer to the mounting base 70.

The mounting base 70 includes a charging pad 72. A power transmitting coil 72A is incorporated in the charging pad 72, and when the power transmitting coil 72A transmits a signal to search for the power receiving coil 66A of the power receiving plate 66 and the power receiving coil 66A is found, a current flows through the power transmitting coil 72A to generate a magnetic field, and the power receiving coil 66A reacts to the magnetic field to start electromagnetic induction. As a result, current flows through the power receiving coil 66A, and power is stored in a battery (not shown) of the smartphone 50 via the USB hub 64.

That is, since the smartphone 50 is automatically charged by placing the stuffed toy 100N as an ornament on the mounting base 70, it is not necessary to take out the smartphone 50 from the space portion 52 of the stuffed toy 100N for charging.

Note that, in the second embodiment, the smartphone 50 is accommodated in the space portion 52 of the stuffed toy 100N and connected by wire (USB connection), but the invention is not limited thereto. For example, a control device having a wireless function (for example, “Bluetooth (registered trademark)”) may be accommodated in the space portion 52 of the stuffed toy 100N, and the control device may be connected to the USB hub 64. In this case, the smartphone 50 and the control device wirelessly communicate with each other without inserting the smartphone 50 into the space portion 52, and the external smartphone 50 is connected to each input/output device via the control device, so that it is possible to provide functions equivalent to those of the robot 100 of the first embodiment. Furthermore, the control device which is accommodated in the space portion 52 of the stuffed toy 100N and the external smartphone 50 may be connected by wire.

Furthermore, although the stuffed bear 100N has been exemplified in the second embodiment, the shape may be another animal, a doll, or a shape of a specific character. Further, the clothes may be changeable. Furthermore, the material of the skin is not limited to the cloth fabric, and may be other materials such as soft vinyl, but is preferably a soft material.

Furthermore, a monitor may be attached to the skin of the stuffed toy 100N, and the control target 252 that provides information to the user 10 through vision may be added. For example, the eyes 56 may be used as a monitor to express joy, anger, sorrow, and pleasure using images projected on the eyes, or a window through which the monitor of the built-in smartphone 50 is transmitted may be provided in the abdomen. Furthermore, the eyes 56 may be used as a projector to express joy, anger, sorrow, and pleasure by using an image projected on a wall surface.

According to the second embodiment, the existing smartphone 50 is placed in the stuffed toy 100N, and the camera 203, the microphone 201, the speaker 60, and the like are extended from the place to appropriate positions via the USB connection.

Further, for wireless charging, the smartphone 50 and the power receiving plate 66 are connected via USB, and the power receiving plate 66 is disposed so as to be as outside as possible when viewed from the inside of the stuffed toy 100N.

In order to use wireless charging of the smartphone 50, it is necessary to arrange the smart phone 50 as outside as possible when viewed from the inside of the stuffed toy 100N, and the stuffed toy 100N is rough when touched from the outside.

Therefore, the smartphone 50 is disposed at the center of the stuffed toy 100N as much as possible, and the wireless charging function (power receiving plate 66) is disposed outside as viewed from the inside of the stuffed toy 100N as much as possible. The camera 203, the microphone 201, the speaker 60, and the smartphone 50 receive wireless power supply via the power receiving plate 66.

Note that other configurations and effects of the stuffed toy 100N of the second embodiment are similar to those of the robot 100 of the first embodiment, and thus the description thereof will be omitted.

Further, a part of the stuffed toy 100N (for example, the sensor module unit 210, the storage unit 220, and the control unit 228) may be provided outside the stuffed toy 100N (for example, the server), and the stuffed toy 100N may function as each part of the stuffed toy 100N by communicating with the outside.

### Third Embodiment

In the first embodiment, the case in which the action control system is applied to the robot 100 has been exemplified, but in the third embodiment, the robot 100 is used as an agent for interacting with a user, and the action control system is applied to an agent system. Note that parts having the same configurations as those of the first and second embodiments are denoted by the same reference numerals, and description thereof is omitted.

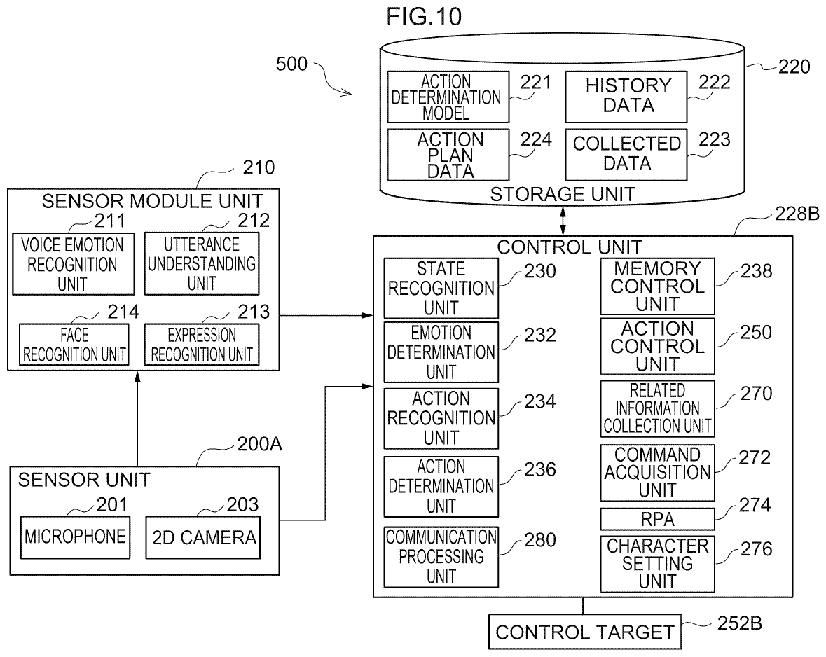

FIG. 10 is a functional block diagram of an agent system 500 configured using some or all of the functions of the action control system.

The agent system 500 is a computer system that performs a series of actions according to the intention of the user 10 through an interaction performed with the user 10. The interaction with the user 10 can be performed by voice or text.

The agent system 500 includes a sensor unit 200A, a sensor module unit 210, a storage unit 220, a control unit 228B, and a control target 252B.

The agent system 500 can be mounted on, for example, a robot, a doll, a stuffed toy, a wearable terminal (pendants, smartwatches, smart glasses), a smartphone, a smart speaker, earphones, a personal computer, or the like. Furthermore, the agent system 500 may be implemented in a web server and used via a web browser operating on a communication terminal such as a smartphone carried by the user.

The agent system 500 serves as, for example, a butler, a secretary, a teacher, a partner, a friend, a lover, or a teacher acting for the user 10. The agent system 500 not only interacts with the user 10 but also provides advice, guides to a destination, gives recommendations according to user's preference, or the like. In addition, the agent system 500 performs reservation, order, payment, or the like to a service provider.

The emotion determination unit 232 determines an emotion of the user 10 and an emotion of the agent itself, similarly in the first embodiment. The action determination unit 236 determines an action of the robot 100 in consideration of emotions of the user 10 and the agent. In other words, the agent system 500 understands the emotion of the user 10 and reads the air to realize heartfelt support, assistance, advice, and service provision. Furthermore, the agent system 500 comforts, encourages, and energizes the user by listening to concerns of the user 10. Furthermore, the agent system 500 plays with the user 10 and draws a picture diary to remind the user of the past. The agent system 500 performs an action that increases the sense of happiness of the user 10. Here, the agent refers to an agent that operates on software.

The control unit 228B includes a state recognition unit 230, an emotion determination unit 232, an action recognition unit 234, an action determination unit 236, a memory control unit 238, an action control unit 250, a related information collection unit 270, a command acquisition unit 272, Robotic Process Automation (RPA) 274, a character setting unit 276, and a communication processing unit 280. The control unit 228B may have the specific processing unit 290 illustrated in FIG. 2B.

As in the first embodiment, the action determination unit 236 determines an utterance content of the agent for interacting with the user 10 as an action of the agent. The action control unit 250 outputs the utterance content of the agent using at least one of voice or text through a speaker or a display that serves as the control target 252B.

The character setting unit 276 sets a character of the agent when the agent system 500 interacts with the user 10 based on designation by the user 10. In other words, the utterance content output from the action determination unit 236 is output through the agent having the set character. As the character, for example, a real famous figure or a famous person such as an actor, an entertainer, an idol, or a sport player can be set. Furthermore, it is also possible to set a fictitious character appearing in a cartoon, a movie, or an animation. In a case in which the character of the agent is known, since the voice, the wording, the tone, and the personality of the character are known, the character setting unit 276 can automatically set prompts only by the user 10 designating his/her favorite character. The voice, wording, tone, and personality of the set character are reflected in the interaction with the user 10. In other words, the action control unit 250 synthesizes a voice corresponding to the character set by the character setting unit 276, and outputs the utterance content of the agent in the synthesized voice. As a result, the user 10 can feel as if he/she is interacting with his/her favorite character (for example, a favorite actor).

In a case in which the agent system 500 is mounted on a device having a display such as a smartphone, for example, an icon, a still image, or a moving image of the agent having a character set by the character setting unit 276 may be displayed on the display. The image of the agent is generated using, for example, an image synthesis technology such as 3D rendering. In the agent system 500, an interaction with the user 10 may be performed while the image of the agent performs a gesture according to the emotion of the user 10, the emotion of the agent, and the utterance content of the agent. Note that the agent system 500 may output only voice without outputting an image when interacting with the user 10.

As in the first embodiment, the emotion determination unit 232 determines an emotion value indicating the emotion of the user 10 and an emotion value of the agent itself. In the present embodiment, the emotion value of the agent is determined instead of the emotion value of the robot 100. The emotion value of the agent itself is reflected in the emotion of the set character. When the agent system 500 interacts with the user 10, not only the emotion of the user 10 but also the emotion of the agent is reflected in the interaction. In other words, the action control unit 250 outputs the utterance content in a mode according to the emotion determined by the emotion determination unit 232.

Furthermore, the emotion of the agent is also reflected in a case in which the agent system 500 performs an action toward the user 10. For example, in a case in which the user 10 requests the agent system 500 to take a photo, whether or not the agent system 500 takes a photo in response to the request from the user is determined according to the degree of “sadness” felt by the agent. In a case in which the character has a positive emotion, the character performs a favorable interaction or action with respect to the user 10, and in a case in which the character has a negative emotion, the character performs a defiant interaction or action with respect to the user 10.

The history data 222 stores a history of the interactions performed between the user 10 and the agent system 500 as event data. The storage unit 220 may be realized by an external cloud storage. In a case of interacting with the user 10 or performing an action toward the user 10, the agent system 500 decides the interaction content or the action content in consideration of the content of the interaction history stored in the history data 222. For example, the agent system 500 grasps hobbies and preferences of the user 10 based on the interaction history stored in the history data 222. The agent system 500 generates an interaction content matching the hobbies and preferences of the user 10 and provides a recommendation. The action determination unit 236 determines the utterance content of the agent based on the interaction history stored in the history data 222. In the history data 222, personal information such as the name, address, telephone number, and credit card number of the user 10 acquired through interactions with the user 10 is stored. Here, an agent may spontaneously make an utterance of inquiry about whether or not to register personal information with the user 10, such as “Do you want me to register your credit card number?”, and the personal information may be stored in the history data 222 according to the answer of the user 10.

As described in the first embodiment, the action determination unit 236 generates the utterance content based on the sentence generated using the sentence generation model. Specifically, the action determination unit 236 inputs the text or voice input by the user 10 and the emotions of both the user 10 and the character determined by the emotion determination unit 232, and the conversation history stored in the history data 222 to the sentence generation model to generate the utterance content of the agent. At this time, the action determination unit 236 may further input the personality of the character set by the character setting unit 276 to the sentence generation model to generate the utterance content of the agent. In the agent system 500, the sentence generation model is not located on the front-end side serving as a touch point for the user 10, but is used solely as a tool of the agent system 500.

The command acquisition unit 272 uses the output of the utterance understanding unit 212 to acquire a command of the agent from a voice or a text uttered from the user 10 through an interaction with the user 10. The command includes, for example, contents of actions to be executed by the agent system 500, such as information search, store reservation, ticket arrangement, purchase of products/services, payment, route guidance to a destination, and recommendation provision.

The RPA 274 performs an action according to the command acquired by the command acquisition unit 272. For example, the RPA 274 performs actions related to use of the service provider, such as information search, store reservation, ticket arrangement, purchase of products/services, and payment.

The RPA 274 reads the personal information of the user 10 necessary for executing the action related to the use of the service provider from the history data 222 and uses the personal information. For example, in a case of purchasing a product in response to a request from the user 10, the agent system 500 reads and uses personal information such as the name, address, telephone number, and credit card number of the user 10 stored in the history data 222. Requesting the user 10 to input personal information in the initial setting is unkind, giving discomfort to the user. In the agent system 500 according to the present embodiment, instead of requesting the user 10 to input personal information in the initial setting, the personal information acquired through interactions with the user 10 is stored, and used by reading if necessary. As a result, it is possible to avoid making the user feel any discomfort, and convenience of the user is improved.

The agent system 500 executes an interactive process by, for example, following steps 1 to 5.

(Step 1) The agent system 500 sets a character of the agent. Specifically, the character setting portion 276 sets a character of the agent when the agent system 500 interacts with the user 10 based on designation by the user 10.

(Step 2) The agent system 500 acquires the state of the user 10 including the voice or text input from the user 10, the emotion value of the user 10, the emotion value of the agent, and the history data 222. Specifically, the process similar to steps S100 to S103 is performed to acquire the state of the user 10 including the voice or text input from the user 10, the emotion value of the user 10, the emotion value of the agent, and the history data 222.

(Step 3) The agent system 500 determines the utterance content of the agent.

Specifically, the action determination unit 236 inputs the text or voice input by the user 10, the emotions of both the user 10, the character determined by the emotion determination unit 232, and the conversation history stored in the history data 222 to the sentence generation model to generate the utterance content of the agent.

For example, the utterance content of the agent is acquired by adding a fixed sentence “At this time, what would you answer as an agent?” to the text or voice input by the user 10, the text indicating the emotions of both the user 10 and the character specified by the emotion determination unit 232 and the conversation history stored in the history data 222, and inputting the fixed sentence to the sentence generation model.

As an example, in a case in which the text or voice input to the user 10 is “I want you to reserve a close nice Chinese restaurant for 7 this evening”, an utterance content of the agent such as “Understood.” and “These are recommendable restaurants. 1.AAAA. 2.BBBB. 3.CCCC. 4.DDDD” is obtained.

Furthermore, in a case in which the text or voice input to the user 10 is “No. 4 DDDD sounds good”, an utterance content of the agent such as “Certainly. I will make a reservation. How many seats?” is obtained.

(Step 4) The agent system 500 outputs the utterance content of the agent.

Specifically, the action control unit 250 synthesizes a voice corresponding to the character set by the character setting unit 276, and outputs the utterance content of the agent in the synthesized voice.

(Step 5) The agent system 500 determines whether or not it is a timing to execute the command of the agent.

Specifically, the action determination unit 236 determines whether or not it is a timing to execute the command of the agent based on the output of the sentence generation model. For example, in a case in which the output of the sentence generation model includes that the agent should execute the command, it is determined that it is the timing to execute the command of the agent, and the process proceeds to step 6. On the other hand, in a case in which it is determined that it is not the timing to execute the command of the agent, the process returns to step 2 described above.

(Step 6) The agent system 500 executes the command of the agent.

Specifically, the command acquisition unit 272 acquires the command of the agent from the voice or text uttered from the user 10 through the interaction with the user 10. Then, the RPA 274 performs an action corresponding to the command acquired by the command acquisition unit 272. For example, in a case in which the command is “information search”, information search is performed by using a search site using a search query obtained through an interaction with the user 10 and an application programming interface (API). The action determination unit 236 inputs the search result to the sentence generation model to generate the utterance content of the agent. The action control unit 250 synthesizes a voice corresponding to the character set by the character setting unit 276, and outputs the utterance content of the agent by using the synthesized voice.

Furthermore, in a case in which the command is “store reservation”, the reservation is made by making a phone call to the store to be reserved using the reservation information obtained through the interaction with the user 10, information of the store to be reserved, and the API using the phone software. At this time, the action determination unit 236 acquires the utterance content of the agent with respect to the voice input from the partner using the sentence generation model having the interaction function. Then, the action determination unit 236 inputs the result of the store reservation (whether or not the reservation is successful) to the sentence generation model to generate the utterance content of the agent. The action control unit 250 synthesizes a voice corresponding to the character set by the character setting unit 276, and outputs the utterance content of the agent by using the synthesized voice.

Then, the process returns to step 2 described above.

In step 6, the result of the action (for example, store reservation) executed by the agent is also stored in the history data 222. The result of the action executed by the agent stored in the history data 222 is used by the agent system 500 to grasp hobbies or preferences of the user 10. For example, in a case in which the same store has been reserved multiple times, it is recognized that the user 10 likes the store, or the reservation details such as the time slot for reservation, or details of the course, or the fee are used as a criterion for choosing the store for reservation of the next time.

In this manner, the agent system 500 can execute the interaction processing and perform an action related to use of the service provider if necessary.

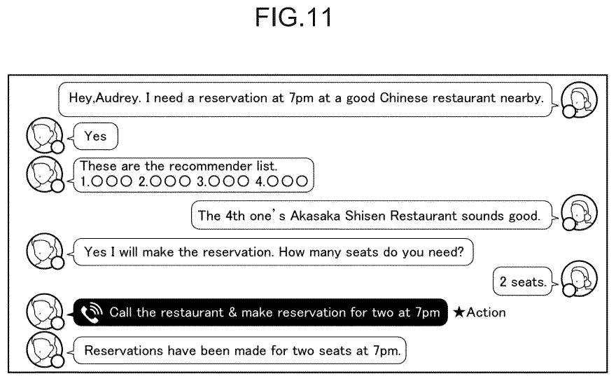

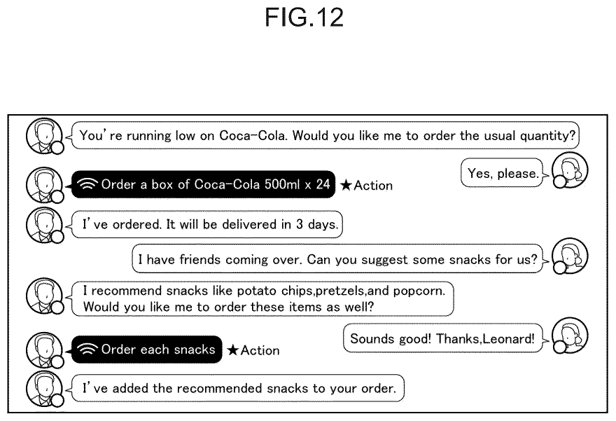

FIG. 11 and FIG. 12 illustrate an example of an operation of the agent system 500. FIG. 11 illustrates a mode in which the agent system 500 makes a restaurant reservation through an interaction with the user 10. In FIG. 11, the utterance contents of the agent are shown on the left side, and the utterance contents of the user 10 are shown on the right side. The agent system 500 can ascertain preferences of the user 10 based on an interaction history with respect to the user 10, provide a list of restaurant recommendations that match the preferences of the user 10, and perform a reservation for a selected restaurant.

Meanwhile, FIG. 12 illustrates a mode in which the agent system 500 accesses an e-commerce site through the interaction with the user 10 to purchase the product. In FIG. 12, the utterance contents of the agent are shown on the left side, and the utterance contents of the user 10 are shown on the right side. The agent system 500 can estimate the remaining amount of the beverage stocked by the user based on the interaction history with respect to the user 10, and can propose purchase of the beverage to the user 10 and execute purchase. Furthermore, the agent system 500 can grasp the preferences of the user based on the past interaction history with respect to the user 10, and recommend a snack that the user likes. In this manner, the agent system 500 supports daily life of the user 10 by performing various actions such as restaurant reservation or product purchase and payment while communicating with the user 10 as an agent such as a butler.

Although the functions of the agent system 500 have been mainly described for the system according to the invention in the above description, the system according to the invention is not necessarily implemented in the agent system. The system according to the invention may be implemented as a general information processing system. The invention may be implemented as, for example, a software program that operates on a server or a personal computer, or an application that operates on a smartphone or the like. The method according to the invention may be provided to a user in a form of software as a Service (SaaS).

Note that other configurations and operations of the agent system 500 of the third embodiment are similar to those of the robot 100 of the first embodiment, and thus description thereof is omitted.

Furthermore, a part of the agent system 500 (for example, the sensor module unit 210, the storage unit 220, and the control unit 228B) may be provided outside a communication terminal such as a smartphone carried by the user (for example, on a server), and the communication terminal may function as each unit of the agent system 500 by communicating with the outside.

### Fourth Embodiment

In a fourth embodiment, the agent system is applied to smart glasses. Note that parts having the same configurations as those of the first to third embodiments are denoted by the same reference numerals, and description thereof is omitted.

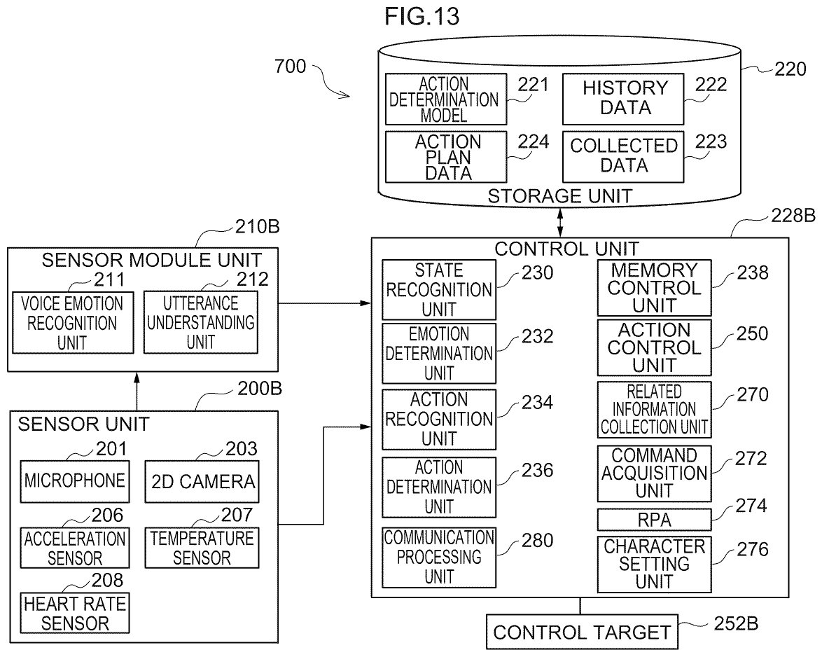

FIG. 13 is a functional block diagram of an agent system 700 configured using some or all of the functions of the action control system. The agent system 700 includes a sensor unit 200B, a sensor module unit 210B, a storage unit 220, a control unit 228B, and a control target 252B. The control unit 228B includes a state recognition unit 230, an emotion determination unit 232, an action recognition unit 234, an action determination unit 236, a memory control unit 238, an action control unit 250, a related information collection unit 270, a command acquisition unit 272, an RPA 274, a character setting unit 276, and a communication processing unit 280. The control unit 228B may have the specific processing unit 290 illustrated in FIG. 2B.

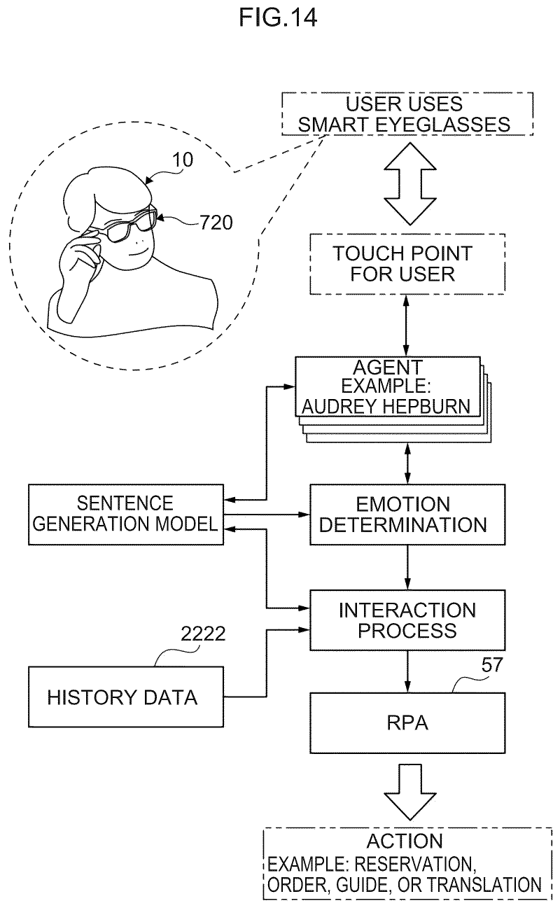

As illustrated in FIG. 14, the smart glasses 720 are a glasses-type smart device, and are worn by the user 10 similarly to general glasses. The smart glasses 720 are an example of electronic equipment and a wearable terminal.

The smart glasses 720 include the agent system 700. The display included in the control target 252B displays various types of information to the user 10. The display is, for example, a liquid crystal display. The display is provided, for example, in a lens portion of the smart glasses 720, and the display content can be visually recognized by the user 10. The speaker included in the control target 252B outputs a voice indicating various types of information to the user 10. The smart glasses 720 include a touch panel (not illustrated), and the touch panel receives inputs from the user 10.

An acceleration sensor 206, a temperature sensor 207, and a heart rate sensor 208 of the sensor unit 200B detect states of the user 10. Note that these sensors are merely examples, and it is a matter of course that other sensors may be mounted to detect states of the user 10.

A microphone 201 acquires voices uttered by the user 10 or environmental sounds around the smart glasses 720. A 2D camera 203 can image the surroundings of the smart glasses 720. The 2D camera 203 is, for example, a CCD camera.

The sensor module unit 210B includes a voice emotion recognition unit 211 and an utterance understanding unit 212. The communication processing unit 280 of the control unit 228B controls communication between the smart glasses 720 and the outside.

FIG. 14 is a diagram illustrating an example of a usage mode of the agent system 700 on the smart glasses 720. The smart glasses 720 realize provision of various services to the user 10 using the agent system 700. For example, when the user 10 operates the smart glasses 720 (for example, sound input to a microphone, or tapping the touch panel with a finger.), the smart glasses 720 start using the agent system 700. Here, using the agent system 700 includes modes in which the smart glasses 720 have the agent system 700 and use the agent system 700, and a part (for example, the sensor module unit 210B, the storage unit 220, and the control unit 228B) of the agent system 700 is provided outside the smart glasses 720 (for example, a server) and the smart glasses 720 communicate with the outside to use the agent system 700.

When the user 10 operates the smart glasses 720, a touch point is generated between the agent system 700 and the user 10. That is, provision of services by the agent system 700 is started. As described in the third embodiment, in the agent system 700, a character of the agent is set by the character setting unit 276.

The emotion determination unit 232 determines an emotion value indicating the emotion of the user 10 and an emotion value of the agent itself. Here, the emotion value indicating the emotion of the user 10 is estimated from various sensors included in the sensor unit 200B mounted on the smart glasses 720. For example, in a case in which a heart rate of the user 10 detected by the heart rate sensor 208 is increased, the emotion values for “anxiety” and “fear” are estimated to be high.

Furthermore, as a result of measuring the body temperature of the user by using the temperature sensor 207, for example, in a case in which the body temperature exceeds the average body temperature, the emotion value for “suffering” or “hardship” is estimated to be high. Furthermore, for example, in a case in which the acceleration sensor 206 detects that the user 10 is playing some kind of sport, the emotion value for “pleasant” is estimated to be large.

Furthermore, for example, the emotion value of the user 10 may be estimated from the voice or utterance content of the user 10 acquired by the microphone 201 mounted on the smart glasses 720. For example, in a case in which the user 10 is raising his/her voice, the emotion value for “anger” is estimated to be high.

In a case in which the emotion value estimated by the emotion determination unit 232 is higher than a predetermined value, the agent system 700 causes the smart glasses 720 to acquire information regarding the surrounding situation. Specifically, for example, the 2D camera 203 is caused to capture an image or a moving image representing the situation around the user 10 (for example, a person or an object around the user). Further, the microphone 201 is caused to record ambient environmental sound. Other examples of the information regarding the surrounding situation include information indicating date, time, positional information, weather, and the like. The information regarding the surrounding situation is stored in the history data 222 together with the emotion value. The history data 222 may be realized by an external cloud storage. As described above, the surrounding situation obtained by the smart glasses 720 is stored in the history data 222 as a so-called life log in a state of being associated with the emotion value of the user 10 at that time.

In the agent system 700, the information indicating the surrounding situation is stored in the history data 222 in association with the emotion value. As a result, the agent system 700 ascertains personal information such as hobbies, preferences, or personality of the user 10. For example, in a case in which an image representing a state of baseball game watching is associated with an emotion value for “joy” or “pleasant”, the hobby of the user 10 is baseball game watching, and the agent system 700 ascertains his/her favorite team or player from the information stored in the history data 222.

Then, in a case of interacting with the user 10 or performing an action toward the user 10, the agent system 700 determines the interaction content or the action content in consideration of the details of the surrounding situations stored in the history data 222. Note that, as a matter of course, the interaction content or the action content may be determined in consideration of the interaction history stored in the history data 222 as described above in addition to the surrounding situations.

As described above, the action determination unit 236 generates the utterance content based on the sentence generated by the sentence generation model. Specifically, the action determination unit 236 inputs the text or voice input by the user 10, the emotions of both the user 10 and the agent determined by the emotion determination unit 232, the conversation history stored in the history data 222, the personality of the agent, and the like to the sentence generation model to generate the utterance content of the agent. Furthermore, the action determination unit 236 inputs the surrounding situations stored in the history data 222 to the sentence generation model to generate the utterance content of the agent.

The generated utterance content is output in voice from a speaker mounted on the smart glasses 720 to the user 10, for example. In this case, a synthesized voice corresponding to the character of the agent is used as the voice. The action control unit 250 generates a synthesized voice by reproducing the voice quality of the character of the agent or generates a synthesized voice according to the emotion of the character (for example, in the case of the emotion “anger”, a voice in a strong tone). Furthermore, the utterance content may be displayed on the display instead of a voice output or together with a voice output.

The RPA 274 executes an operation according to a command (for example, a command of the agent acquired from a voice or text uttered by the user 10 through interactions with the user 10.). The RPA 274 performs actions related to use of service providers, such as information search, store reservation, ticket arrangement, purchase of products/services, payment, route guidance, and translation.

Furthermore, as another example, the RPA 274 executes an operation of transmitting a content input by voice of the user 10 (for example, a child) through interactions with the agent to the other party (for example, the parent). Examples of the transmission means include message application software, chat application software, mail application software, and the like.

In a case in which the operation by the RPA 274 is executed, for example, a voice indicating that the execution of the operation has been finished is output from a speaker mounted on the smart glasses 720. For example, a voice such as “Reservation for the store has been completed” is output to the user 10. Furthermore, for example, in a case in which reservation of the store is full, a voice indicating “Reservation could not be made. What would you like to do?” is output to the user 10.

In a case in which the control unit 228B includes the specific processing unit 290, the specific processing unit 290 performs the same specific process as that of the third embodiment and controls an action of the agent so as to output a result of the specific process. At this time, an utterance content of the agent for interacting with the user 10 is determined as the action of the agent, and the utterance content of the agent is output by a speaker or a display as the control target 252B in at least one of voice or text.

Note that the smart glasses 720 may function as each unit of the agent system 700 when some units of the agent system 700 (for example, the sensor module unit 210B, the storage unit 220, and the control unit 228B) are provided outside the smart glasses 720 (for example, a server), and the smart glasses communicate with the outside.

As described above, with the smart glasses 720, various services are provided to the user 10 by using the agent system 700. In addition, since the smart glasses 720 are worn by the user 10, the agent system 700 can be used in various scenes such as at home, at work, and at a place outside the house.

In addition, since the smart glasses 720 are worn by the user 10, the smart glasses are suitable for collecting so-called life logs of the user 10. Specifically, an emotion value of the user 10 is estimated based on detection results by various sensors or the like mounted on the smart glasses 720 or recording results of the 2D camera 203 or the like. Therefore, emotion values of the user 10 can be collected in various scenes, and the agent system 700 can provide a service or utterance content suitable for the emotions of the user 10.

Furthermore, in the smart glasses 720, situations around the user 10 can be obtained by the 2D camera 203, the microphone 201, and the like. Then, these surrounding situations and the emotion values of the user 10 are associated with each other. As a result, it is possible to estimate what kind of emotion the user 10 has in what kind of situation. As a result, the accuracy in the agent system 700 to ascertain the hobbies/preferences of the user 10 can be improved. Then, in the agent system 700, the hobbies/preferences of the user 10 are accurately ascertained, and thereby the agent system 700 can provide a service or an utterance content suitable for the hobbies/preferences of the user 10.

Furthermore, the agent system 700 can also be applied to other wearable terminals (electronic equipment that can be worn on the body of the user 10, such as a pendant, a smart watch, an earring, a bracelet, or a hairband.). In a case in which the agent system 700 is applied to a smart pendant, a speaker as the control target 252B outputs a voice indicating various types of information to the user 10. The speaker is, for example, a speaker capable of outputting a voice having directivity. The speaker is set to have directivity toward the ears of the user 10. As a result, the voice is prevented from reaching a person other than the user 10. The microphone 201 acquires a voice uttered by the user 10 or an environmental sound around the smart pendant. The smart pendant is worn in such a way that it hangs around the neck of the user 10. Thus, the smart pendant is located relatively close to the mouth of the user 10 while being worn. This facilitates acquisition of voices uttered by the user 10.

### Fifth Embodiment

In a fifth embodiment, the robot 100 is applied as an agent for interacting with a user through an avatar. That is, the action control system is applied to an agent system configured using a headset-type terminal. Note that parts having the same configurations as those of the first and second embodiments are denoted by the same reference numerals, and description thereof is omitted.

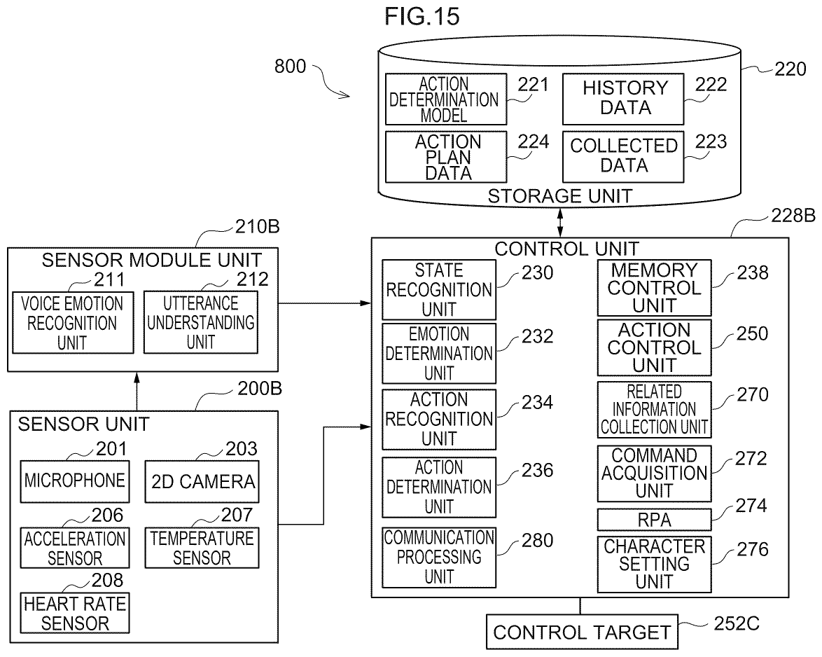

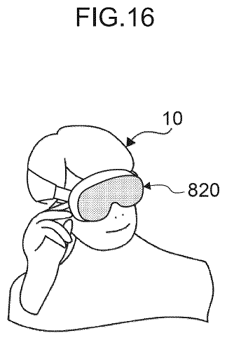

FIG. 15 is a functional block diagram of an agent system 800 configured using some or all of the functions of the action control system. The agent system 800 includes a sensor unit 200B, a sensor module unit 210B, a storage unit 220, a control unit 228B, and a control target 252C. The agent system 800 is implemented by, for example, a headset-type terminal 820 as illustrated in FIG. 16. The control unit 228B may have the specific processing unit 290 illustrated in FIG. 2B.

Further, the headset-type terminal 820 may function as each unit of the agent system 800 when a part of the headset-type terminal 820 (for example, the sensor module unit 210B, the storage unit 220, and the control unit 228B) is provided outside the headset-type terminal 820 (for example, a server) and the headset-type terminal communicates with the outside.

In the embodiment, the control unit 228B has the functions of determining an action of the avatar and generating display of the avatar to be presented to the user through the headset-type terminal 820.

As in the first embodiment, the emotion determination unit 232 of the control unit 228B determines an emotion value of the agent based on the state of the headset-type terminal 820, and substitutes the emotion value as an emotion value of the avatar.

When the avatar performs a response process of responding to an action of the user 10 as in the first embodiment, the action determination unit 236 of the control unit 228B determines an action of the avatar based on at least one of a user state, a state of the headset-type terminal 820, an emotion of the user, or an emotion of the avatar.

As in the first embodiment, when an agent functioning as an avatar performs an autonomous process of autonomously acting, the action determination unit 236 of the control unit 228B determines, as an action of the avatar, any of multiple types of avatar actions including not acting, using at least one of the state of the user 10, the emotion of the user 10, the emotion of the avatar, or the state of electronic equipment (for example, the headset-type terminal 820) that controls the avatar, and the action determination model 221, at a predetermined timing.

Specifically, the action determination unit 236 inputs a text representing at least one of the state of the user 10, the state of the electronic equipment, the emotion of the user 10, or the emotion of the avatar, together with a text for inquiry about the action of the avatar to the sentence generation model, and determines the action of the avatar based on the output of the sentence generation model. The multiple types of avatar actions include (1) to (16), as in the first embodiment.

In a case in which the action determination unit 236 determines, as an avatar action, that “(12) Prepare minutes.”, that is, preparing minutes, the action determination unit prepares the meeting minutes and summarizes the meeting minutes using the sentence generation model. To perform the summarization, the action determination unit 236 may cause the avatar to output a voice such as “prepare minutes” through a speaker or to display a text in an image display area of the headset-type terminal 820. Note that the action determination unit 236 may prepare minutes without using an avatar action.

In addition, with respect to “(12) Prepare minutes”, the memory control unit 238 stores the created summary in the history data 222. Further, the memory control unit 238 detects the utterance of each of the participants in the meeting, as a state of the user, using the microphone function of the headset-type terminal 820 and stores the utterances in the history data 222. Here, although the creation and summarization of the minutes are autonomously performed with a predetermined trigger, for example, a trigger such as an end of a meeting, the configuration is not limited thereto, and the creation and summarization may be performed in the middle of the meeting. Furthermore, the summary of the minutes is not limited to the case of using the sentence generation model, and other known methods may be used.

In a case in which “(13) Gives advice on the user utterance”, that is, an output of advice information on the user utterance at the meeting, is determined as an avatar action, the action determination unit 236 determines the advice using the data generation model based on the summary stored in the history data 222 and outputs the advice. In the output of the advice, it is desirable to control the avatar such that the avatar is speaking as described below. Here, the case in which an output of advice information is determined includes a case in which a relationship with the stored summaries of the past meetings, for example, similar speech, is made, and the determination is autonomously performed. In addition, the determination as to whether the utterance is similar is performed using, for example, a known method of converting the utterance into a vector (numerical value) and calculating a similarity between the vectors, but may be performed using another method. Note that, materials of the meeting may be input into the data generation model in advance, and as terms described in the materials are expected to appear frequently, the terms may be excluded from the detection of similar utterances.

In addition, the advice information includes advice for meeting participants participating in the meeting wearing the headset-type terminal 820 based on the results of comparisons with past meetings, including spontaneous remarks such as, “That content was already presented by someone on the date” or “This content is superior to the person's proposal in this respect”. Further “(13) Gives advice on the user speech” includes user speech in a meeting different from the meeting for which the summary was created according to “(12) Prepare minutes” described above. That is, whether similar speech was made in a past meeting is determined and the advice information is output.

In a case in which the action determination unit 236 determines “(14) Support the progress of the meeting” as an avatar action, that is, the meeting is in a predetermined state, the avatar spontaneously supports the progress of the meeting. Here, support for the progress of the meeting includes actions to summarize the meeting, for example, organizing frequently used terms, uttering a summary of previous meetings, and actions to help participants clear their heads, for instance, by offering alternative topics. By performing such actions, the progress of the meeting can be supported. In the output of the support for the progress of the meeting, it is desirable to control the avatar such that the avatar is speaking as described below. Here, the case in which the meeting has reached a predetermined state includes a state in which speech is no longer accepted for a predetermined time. That is, in a case in which multiple users do not speak for a predetermined period of time, that is, for 5 minutes, it is determined that the meeting has reached a deadlock, no good ideas are emerging, and a state of silence has fallen. Thus, the meeting is summarized by compiling frequently used words. Furthermore, a case in which a meeting has reached a predetermined state includes a state in which a term included in speech is received a predetermined number of times. That is, in a case in which the same term is received a predetermined number of times, it is determined that the same topic is going around in the meeting and no new ideas are coming out. Thus, the meeting is summarized by compiling frequently used words. Note that, materials of the meeting may be input into the sentence generation model in advance, and as terms described in the materials are expected to appear frequently, the terms may be excluded from counting the number of times.

In a case in which the action determination unit 236 determines “(15) Take meeting minutes” as an action of the avatar corresponding to an action of the user 10, the action determination unit acquires the utterance content of the user 10 by voice recognition, identifies the speaker by voiceprint authentication, acquires the emotion of the speaker based on the determination result of the emotion determination unit 232, and creates minutes data representing a combination of the utterance of the user 10, the identification result of the speaker, and the emotion of the speaker. The action determination unit 236 generates a summary of the text representing the minutes data by using a sentence generation model having an interaction function. The action determination unit 236 further generates a list of things that the user should do (to-do list) included in the summary by using the sentence generation model having an interaction function. This to-do list includes at least a person in charge (responsible person), an action content, and the deadline for each thing the user should do. The action determination unit 236 further transmits the minutes data, the summary, and the to-do list to the participants of the meeting. The action determination unit 236 further transmits a message to confirm things to do to the person in charge before the predetermined number of days determined by the deadline based on the person in charge and the deadline included in the list.

Specifically, when the user 10 speaks “take the minutes”, the action determination unit 236 determines to take the meeting minutes as an action corresponding to the action of the user 10. Thereby, the minutes data including the information indicating the person who spoke can be obtained. When the user 10 speaks “send the summary to the persons concerned” at the end of the meeting, the action determination unit 236 summarizes the meeting minutes, creates a to-do list, and transmits the summary and the to-do list to the persons concerned.

When summarizing the meeting minutes, the text of the created minutes data and a fixed sentence “Summarize the content” are input to a generative AI that is the sentence generation model, and a summary of the meeting minutes is acquired. Furthermore, when creating a to-do list, a text of the summary of meeting minutes and a fixed sentence “create a to-do list” are input to the generative AI that is a sentence generation model, and thereby a to-do list is acquired. As a result, upon understanding the content of the meeting, the meeting can be summarized, and thereby a to-do list can be created and the responsible parties for the to-do list can be organized. Categorization of the to-do list is performed by authenticating a voiceprint to recognize the person who spoke. Based on the determination result of the emotion determination unit 232, it is possible to combine the evaluation as to whether a person is reluctantly motivated to do something or is enthusiastically attempting to do something. It is possible to identify who will do what by when. In a case in which the person in charge, the deadline, and the like are not determined, an utterance of making an inquiry to the user 10 may be determined as an action of the avatar. As a result, a message like “The person in charge for AAA has not been decided yet. Who would do it?” can be uttered by the avatar.

Note that features related to the date and time may be extracted from the summary of the meeting minutes to register the features on a calendar or create a to-do list.

Furthermore, the action determination unit 236 may further determine, as an action of the avatar, to utter the conclusion or a summary of the meeting at the end of the meeting. In addition, the action determination unit 236 transmits the minutes data, the summary, and the to-do list to the participants of the meeting. The action determination unit 236 also sends a reminder of the to-do list to the person in charge.

As an example, in a case in which the action determination unit 236 determines to take the meeting minutes as an action corresponding to the action of the user 10, the action determination unit performs the processing of step 1 to step 9 below as in the first embodiment.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice.

In particular, in a case in which the action determination unit 236 determines to produce and play music in consideration of an event on the previous day as an action of the avatar, the action control unit 250 controls the avatar to play the music by performing or singing the music, for example. That is, in a case in which the action determination unit 236 determines to produce and play music in consideration of an event on the previous day as an action of the avatar, the action determination unit 236 selects the event data of that day from the history data 222 at the end of one day and reviews all the conversation contents and the event data of that day, as in the first embodiment. The action determination unit 236 adds a fixed sentence “Summarize this content” to the text indicating the reviewed content and inputs the text to the sentence generation model, thereby acquiring a summary of the history of the previous day. The summary reflects the action and emotion of the user 10 on the previous day, and further the action and emotion of the avatar. The summary is stored in, for example, the storage unit 220. The action determination unit 236 acquires the summary of the previous day in the next morning, inputs the acquired summary to the music generation engine, and acquires music summarizing the history of the previous day. As a result, for example, in a case in which the emotion of the avatar is “joyful”, music with a warm atmosphere is acquired, and in a case in which the emotion of the avatar is “angry”, music with a violent atmosphere is acquired.

The action control unit 250 generates an image in which the avatar is performing or singing the music acquired by the action determination unit 236 on a stage in a virtual space. As a result, in the headset-type terminal 820, a state in which the avatar is performing or singing the music is displayed in the image display area. As a result, even if the user 10 and the avatar are not talking with each other, it is possible to spontaneously change the music performed or sung by the avatar based on only the emotion of the user and the emotion of the avatar, so it is possible to make the user feel as if the avatar is alive.

At this time, the action control unit 250 may change the expression of the avatar or change the motion of the avatar according to the content of the summary. For example, in a case in which the content of the summary is a pleasant content, the expression of the avatar may be changed to an expression of pleasure, or the motion of the avatar may be changed as if the avatar is dancing with pleasure. Furthermore, the action control unit 250 may transform the avatar in accordance with the content of the summary. For example, the action control unit 250 may transform the avatar so as to imitate a character in the summary, or transform the avatar so as to imitate an animal, an object, or the like appearing in the summary.

Furthermore, the action control unit 250 may generate an image so as to cause the avatar to have a tablet terminal drawn in a virtual space and perform an operation of transmitting music from the tablet terminal to the terminal device of the user. In this case, by actually transmitting music from the tablet terminal to the mobile terminal device of the user 10, it is possible to express an operation such as transmission of music by e-mail from the tablet terminal to the mobile terminal device of the user 10 or transmission of music to a messenger application as if the avatar is performing the operation. Furthermore, in this case, the user 10 can play and listen to the music on his/her mobile terminal device.

Furthermore, in a case in which the action determination unit 236 determines, as an action of the avatar, to output advice information on the user speech in the meeting, it is preferable to cause the action control unit 250 to control the avatar to output advice information according to the content of the speech. At this time, the action control unit 250 causes a voice of the determined advice to be output from a speaker included in the headset-type terminal 820 or a speaker connected to the headset-type terminal 820 in accordance with the motion of the mouth of the avatar as the avatar is speaking, or causes a text to be displayed and output in the image display area of the headset-type terminal 820.

Furthermore, it is desirable that the output of the advice using the avatar described above by the action determination unit 236 be autonomously executed by the action determination unit 236, instead of being initiated by an inquiry from the user. Specifically, in a case in which a similar utterance has been made, the action determination unit 236 may output the advice information by itself.

Furthermore, in a case in which the action determination unit 236 determines, as an action of the avatar, to output the advice information for the utterance of the user during the meeting, the advice to be output may be determined further based on the state of the headset-type terminal 820 of another user or an emotion of another avatar displayed on the headset-type terminal 820 of the other user. For example, in a case in which the emotion of another avatar is a state of excitement, advice on inducing calm discussion may be output.

Furthermore, as in the first embodiment, the action determination unit 236 may spontaneously and periodically detect a state of the user. Actions of the avatar include output of a summary of events of the previous day through utterance or gesture. In a case in which the action determination unit 236 determines, as an action of the avatar, to output a summary of events of the previous day through utterance or gesture, the action determination unit acquires a summary of event data of the previous day stored in the history data when detecting a predetermined conversation or gesture by the user. The action control unit 250 controls the avatar to output the acquired summary through utterance or gesture.

Specifically, the action determination unit 236 adds a fixed sentence instructing to summarize an event of the previous day into a text representing the event data of the previous day, inputs the text to the sentence generation model which is an example of the action determination model 221, and generates the summary based on the output of the sentence generation model. For example, the event data of that day is selected from the history data 222 at the end of one day, and all the conversation contents and event data of that day are reviewed. The action determination unit 236 adds a fixed sentence “Summarize this content” to the text indicating the reviewed content and inputs the text to the sentence generation model, and thereby acquires a summary of the history of the previous day. The summary reflects the action and emotion of the user 10 on the previous day, and further the action and emotion of the avatar. The summary is stored in, for example, the storage unit 220.

Furthermore, the conversation or gesture predetermined by the user is a conversation in which the user tries to remember the event on the previous day or a gesture in which the user thinks about something. For example, in a case in which a conversation of the user such as “What did you do yesterday?” or a gesture of the user in which the user thinks about something is detected as an example when the system is activated or the user wakes up in the next morning, the action determination unit 236 acquires the summary of the previous day from the storage unit 220. The action control unit 250 controls the avatar to spontaneously output the acquired summary through utterance or gesture.

The action control unit 250 controls the avatar to utter the summary acquired by the action determination unit 236 in the virtual space or express the summary by a gesture. As a result, in the headset-type terminal 820, an appearance of the avatar representing the summary through utterance or a gesture in the image display area is displayed. The user 10 can grasp the outline of the event of the previous day from the utterance or gesture of the avatar.

At this time, the action control unit 250 may change the expression of the avatar or change the motion of the avatar according to the content of the summary. For example, in a case in which the content of the summary is a pleasant content, the expression of the avatar may be changed to an expression of pleasure, or the motion of the avatar may be changed as if the avatar is dancing with pleasure. Furthermore, the action control unit 250 may transform the avatar in accordance with the content of the summary. For example, the action control unit 250 may transform the avatar so as to imitate a character in the summary, or transform the avatar so as to imitate an animal, an object, or the like appearing in the summary.

Furthermore, as in the first embodiment, the action determination unit 236 may spontaneously and periodically detect a state of the user. The action of the avatar includes reflecting an event of the previous day in the emotion of the next day. In a case in which the action determination unit 236 determines to reflect the event of the previous day in the emotion of the next day as an action of the avatar, the action determination unit acquires a summary of the event data of the previous day stored in the history data, and determines the emotion to be held on the next day based on the summary. The action control unit 250 controls the avatar to express the determined emotion to be held on the next day.

Specifically, the action determination unit 236 adds a fixed sentence instructing to summarize an event of the previous day into a text representing the event data of the previous day, inputs the text to the sentence generation model which is an example of the action determination model 221, and generates the summary based on the output of the sentence generation model. For example, the event data of that day is selected from the history data 222 at the end of one day, and all the conversation contents and event data of that day are reviewed. The action determination unit 236 adds a fixed sentence “Summarize this content” to the text indicating the reviewed content and inputs the text to the sentence generation model, and thereby acquires a summary of the history of the previous day. The summary reflects the action and emotion of the user 10 on the previous day, and further the action and emotion of the avatar. The summary is stored in, for example, the storage unit 220.

Then, the action determination unit 236 adds a fixed sentence for asking about an emotion to be held on the next day to the generated text representing the summary, inputs the text to the sentence generation model, and determines the emotion to be held on the next day based on the output of the sentence generation model. For example, a fixed sentence “What emotion should I have tomorrow?” is added to the text representing the summary of the event of the previous day and input to the sentence generation model, and the emotion of the avatar based on the summary of the previous day is determined. That is, the emotion of the avatar will be carried over from the emotion of the previous day. The avatar can start a new day by spontaneously carrying over the emotion of the previous day on the next day.

The action control unit 250 controls the avatar to utter the emotion of the avatar determined by the action determination unit 236 in the virtual space or to express the emotion through a gesture. As a result, in the headset-type terminal 820, an appearance of the avatar representing the emotion of the avatar through utterance or a gesture in the image display area is displayed. For example, if the emotion of the avatar of the previous day is pleasant, the emotion of the avatar will be carried over as pleasure on the next day.

At this time, the action control unit 250 may change the expression of the avatar or change the motion of the avatar according to the content of the emotion that the avatar has. For example, in a case in which the emotion of the avatar is a pleasant emotion, the expression of the avatar may be changed to an expression of pleasure, or the motion of the avatar may be changed as if the avatar dances with pleasure.

Furthermore, in a case in which the action determination unit 236 determines, as an action of the avatar, to output support for the progress of the meeting to the user in the meeting, it is preferable for the action control unit 250 to control the avatar to output support for the progress of the meeting. At this time, the action control unit 250 vocalizes the determined support for the progress and causes the voice to be output from a speaker included in the headset-type terminal 820 or a speaker connected to the headset-type terminal 820 in accordance with the motion of the mouth of the avatar as the avatar is speaking, or causes a text to be displayed and output near the mouth of the avatar in the image display area of the headset-type terminal 820.

With such a configuration, even in a deadlock meeting, it is possible to support the progress of the meeting by summarizing the meeting.

Furthermore, it is desirable that the support for the progress of the meeting using the avatar described above by the action determination unit 236 be autonomously executed by the action determination unit 236, instead of being initiated by an inquiry from the user. Specifically, in a case in which the meeting is in a predetermined state, the action determination unit 236 may perform support for the progress of the meeting by itself.

Furthermore, in a case in which the action determination unit 236 determines, as an action of the avatar, to output support for the progress of the meeting to the user in the meeting, the action determination unit may cause the avatar to operate to determine the content of the support for the progress further based on the state of the headset-type terminal 820 of another user or the emotion of another avatar displayed on the headset-type terminal 820 of the other user. For example, in a case in which the emotion of another avatar is a state of excitement, advice on inducing calm discussion may be output.

Furthermore, in a case in which the action determination unit 236 determines to take minutes as an action of the avatar, the action determination unit may cause the avatar to operate to output a summary of a text representing minutes data with an expression corresponding to the emotion of the speaker. For example, in a case in which the avatar is caused to utter a summary of a text representing minutes data, the avatar is caused to operate with an expression corresponding to the emotion of the speaker corresponding to the utterance content.

Furthermore, in a case in which it is determined to take minutes as an action of the avatar, the action determination unit 236 may cause the avatar to operate in accordance with what the user should do when outputting a list of things that the user should do. For example, in a case in which the avatar is caused to utter a list of things that the user should do, the avatar is caused to operate with a motion corresponding to the things that the user should do. As an example, in a case in which what the user should do is create a document, the avatar is caused to operate the personal computer.

In a case in which the action determination unit 236 determines “(15) The avatar takes meeting minutes” as an avatar action, the action determination unit 236 performs processing similar to the case in which taking meeting minutes is determined as the action of the avatar corresponding to the action of the user 10 described in the above response process.

In addition, the action determination unit 236 may acquire the history data 222 of the user 10 designated from the storage unit 220, and output the content of the acquired history data 222 in a first text file. Furthermore, the action determination unit 236 may acquire the history data 222 of the previous day of the user 10.

The action determination unit 236 adds, to the first text file, an instruction for causing the sentence generation model to summarize the history of the user 10 described in the first text file, for example, “Summarize the contents of this history data!”. A sentence representing the instruction is stored in the storage unit 220 in advance as a fixed sentence, for example, and the action determination unit 236 adds the fixed sentence indicating the instruction to the first text file.

When the action determination unit 236 inputs the first text file to which the fixed sentence indicating the instruction has been added to the sentence generation model, the summary sentence of the history of the user 10 is obtained as an answer from the sentence generation model from the history data 222 of the user 10 described in the first text file.

Furthermore, the action determination unit 236 inputs the summary sentence of the history of the user 10 acquired from the sentence generation model to the image generation model that generates an image associated with the input sentence.

As a result, the action determination unit 236 acquires the summary image visualizing the content of the summary sentence of the history of the user 10 from the image generation model.

Furthermore, the action determination unit 236 outputs the contents of the action of the user 10 stored in the history data 222, the emotion of the user 10 determined from the action of the user 10, and the emotion of the avatar determined by the emotion determination unit 232, and further, a summary sentence (if any) of the history of the previous day of the user 10, in a second text file. In this case, the action determination unit 236 adds a fixed sentence expressed by predetermined words for asking about an action to be taken by the avatar, such as “What action should the avatar take at this time?”, to the second text file expressing the action of the user 10, the emotion of the user 10, the emotion of the avatar, and further a summary sentence (if applicable) of the history of the previous day of the user 10 in characters.

The action determination unit 236 inputs the second text file to which the fixed sentence is added, the summary image, and the summary sentence to the sentence generation model if necessary.

As a result, an action to be taken by the avatar determined based on the action of the user 10, the emotion of the user 10, the emotion of the avatar, and further the information obtained from the summary image and the summary sentence is obtained as an answer from the sentence generation model.

The action determination unit 236 generates an action content of the avatar and determines an action of the avatar according to the content of the answer obtained from the sentence generation model.

Furthermore, the action control unit 250 operates the avatar according to the determined action of the avatar, and displays the avatar in the image display area of the headset-type terminal 820 as the control target 252C. Furthermore, in a case in which the determined action of the avatar includes the utterance content of the avatar, the action control unit 250 outputs the utterance content of the avatar by voice through a speaker as the control target 252C.

In particular, in a case in which it is determined to make an utterance regarding the action history of the user 10 as an action of the avatar, the action determination unit 236 determines to make an utterance regarding the degree of stress of the user 10.

For example, the action determination unit 236 determines to provide a topic related to the degree of stress of the user 10 using the avatar saying “You were unusually irritated yesterday”. The utterance content of the avatar determined by the action determination unit 236 may be a topic related to the cause of stress held by the user 10. At this time, the action control unit 250 causes a speaker included in the control target 252 to output a voice representing the determined utterance content of the avatar.

Furthermore, the action determination unit 236 may select a video that reduces the stress of the user 10 according to the stress level of the user 10, and make a determination to cause the action control unit 250 to display the selected video in the image display area of the headset-type terminal 820. In this case, the action determination unit 236 may determine to change the appearance of the avatar in accordance with the content of the selected video.

For example, in a case in which the selected video is a video of the seaside, the action determination unit 236 changes the avatar to a person in a swimsuit. Furthermore, in a case in which the selected video is a video of a soccer game that is a hobby of the user 10, the avatar explaining the game content is changed to the appearance of the favorite player of the user 10. The avatar does not necessarily have to look like a human, and may be an animal or an article.

Furthermore, the action determination unit 236 may determine to cause the avatar to utter advice for preventing the user 10 from holding stress. For example, the action determination unit 236 may determine, as an action of the avatar, an action of recommending the user 10 to play a sport or recommending the user to go to a museum. In a case in which the user 10 says that he/she wants to go to a museum, the avatar notifies the user of the content of an exhibit being held in the museum. In a case in which the user 10 specifies a museum to which the user 10 wants to go, the action determination unit 236 may determine to display a route from the position of the user to the museum in the image display area of the headset-type terminal 820, and to cause the avatar to utter information such as the opening hours and regular holidays of the museum to the user 10.

Furthermore, in a case in which the person who has caused stress (referred to as a “target person”) can be specified from the action history of the user 10, the action determination unit 236 may display an avatar that looks like the target person in the image display area of the headset-type terminal 820, and determine to take an action of moving the avatar along a story in which the avatar of the target person and the avatar of the user 10 fight each other and finally the avatar of the user 10 wins, or an action of the avatar of the target person making an apology.

Furthermore, the action determination unit 236 may grasp refrigerated and frozen foods purchased by the user and refrigerated and frozen foods consumed by the user from the action history of the user 10, and determine an action of causing the avatar that has transformed into the refrigerator to speak about the foods in the refrigerator as an action of the avatar. In this case, the action determination unit 236 may cause the avatar that has transformed into the refrigerator to open the door of the refrigerator played by the avatar and cause the avatar to take an action to display the contents of the refrigerator to the user 10. As a result, the user 10 can check whether the user has forgotten to buy at the time of shopping.

In a case in which the control unit 228B includes the specific processing unit 290 in the fifth embodiment, as in the first embodiment, the specific processing unit 290 performs processing (specific processing) of acquiring and outputting a response regarding a presentation content in a meeting (one-on-one meeting as an example) that is periodically held, in which one of the users participates as a participant, for example. The action of the avatar includes acquiring and outputting a response related to the presentation content of the meeting. The specific processing unit 290 controls electronic equipment (for example, the headset-type terminal 820) such that a result of the specific processing is output as an action of the avatar.

In the specific process related to the meeting, a condition for a presentation content to be presented by a subordinate at the meeting is set as a predetermined trigger condition, as in the first embodiment. In a case in which a user input satisfies this condition, the specific processing unit 290 uses the output of the sentence generation model when the information obtained from the user input is an input sentence, and acquires and outputs a response to the presentation content of the meeting as a result of the specific process.

Also in the fifth embodiment, the specific processing unit 290 includes an input unit 292, a processing unit 294, and an output unit 296 (see FIG. 2C). The input unit 292, the processing unit 294, and the output unit 296 function and operate as those in the first embodiment. In particular, the processing unit 294 of the specific processing unit 290 performs a specific process using the sentence generation model, for example, a process similar to the example of the operation flow shown in FIG. 4C.

In the fifth embodiment, the output unit 296 of the specific processing unit 290 controls actions of the avatar so as to output the results of the specific process. Specifically, the control is performed such that the summary and the appeal points acquired by the processing unit 294 of the specific processing unit 290 are displayed to the avatar, or the avatar speaks about the summary and the appeal points, or transmits a message indicating the summary and the appeal points to the user of the message application of the user's mobile terminal.

In the fifth embodiment, an action of the avatar can be changed according to the result of the specific process. For example, in a case in which the user faces a supervisor in a one-on-one meeting, it is possible to indicate an actual motion of the subordinate in response to the result of the specific process. As an example, in a case in which the subordinate explains an appeal point to the supervisor, the avatar indicates intonation of the utterance, an expression at the time of the utterance, a gesture, and the like. More specifically, in the case of describing an appeal point at which a high evaluation is obtained from the supervisor, the intonation of the utterance is increased, the expression of the avatar is changed into a proud expression, or the like. Then, in a case in which an actual one-on-one meeting is performed, the user 10 can perform an effective one-on-one meeting by referring to these motions indicated by the avatar.

In the fifth embodiment, not only the avatar as the subordinate who is the user 10 but also the avatar of the supervisor may be displayed. By reproducing the state in which the subordinate and the supervisor face each other using avatars, it is possible to perform a rehearsal of the one-on-one meeting with realistic feeling.

The fifth embodiment can be applied to any user 10 who will participate in a meeting without limitation. For example, the user may be a user 10 who participates in a meeting between “co-workers” in an equal relationship, in addition to a subordinate in a relationship between a supervisor and a subordinate. Furthermore, the user 10 is not limited to a person belonging to a specific organization, and may be a user 10 who holds a meeting.

In the fifth embodiment, it is possible to efficiently prepare for a meeting and implement the meeting for the user 10 who will participate in the meeting. Furthermore, the user 10 can shorten the time for preparing for a meeting and duration in which a meeting is held.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

Note that, although the case in which the headset-type terminal 820 is used has been described as an example in the above embodiment, the invention is not limited thereto, and an eyeglass-type terminal having an image display area for displaying an avatar may be used.

Furthermore, although the case in which the sentence generation model capable of generating a sentence according to input texts is used has been described as an example in the above embodiment, the invention is not limited thereto, and a data generation model other than the sentence generation model may be used. For example, a prompt including an instruction is input to the data generation model, and inference data such as voice data indicating a voice, text data indicating a text, and image data indicating an image is input thereto. The data generation model infers the input inference data according to the instruction indicated by the prompt, and outputs the inference result in a data format such as voice data and text data. Here, the inference refers to, for example, analysis, classification, prediction, and/or summary.

Furthermore, although the case in which the robot 100 recognizes the user 10 using a face image of the user 10 has been described in the above embodiment, the disclosed technology is not limited to this mode. For example, the robot 100 may recognize the user 10 using a voice uttered by the user 10, a mail address of the user 10, an ID of an SNS of the user 10, an ID card carried by the user 10 in which a wireless IC tag is built, or the like.

The robot 100 is an example of electronic equipment including an action control system. The application target of the action control system is not limited to the robot 100, and the action control system can be applied to various types of electronic equipment. Furthermore, the function of the server 300 may be implemented by one or more computers.

At least some functions of the server 300 may be implemented by a virtual machine. Furthermore, at least some functions of the server 300 may be implemented in a cloud.

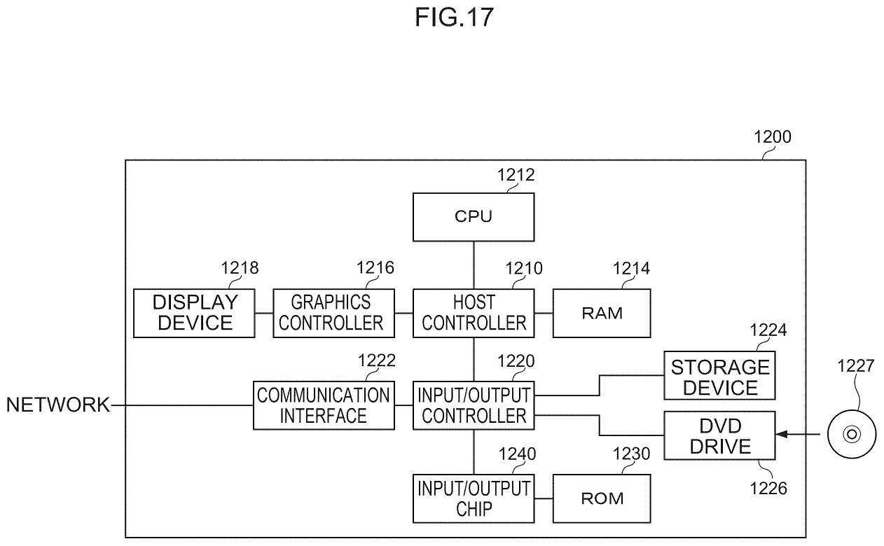

FIG. 17 schematically illustrates an example of a hardware configuration of a computer 1200 functioning as the smartphone 50, the robot 100, the server 300, and the agent systems 500, 700, and 800. A program installed in the computer 1200 can cause the computer 1200 to function as one or more “units” of a device according to the present embodiment, or cause the computer 1200 to execute an operation associated with the device according to the present embodiment or one or more “units” thereof, and/or cause the computer 1200 to execute a process according to the present embodiment or stages of the process. Such programs may be executed by a CPU 1212 to cause the computer 1200 to perform certain operations associated with some or all of the blocks in the flowcharts and block diagrams described in the present specification.

The computer 1200 according to the present embodiment includes the CPU 1212, a RAM 1214, and a graphic controller 1216, which are mutually connected by a host controller 1210. The computer 1200 also includes input/output units such as a communication interface 1222, a storage device 1224, a DVD drive 1226, and an IC card drive, which are connected to the host controller 1210 via an input/output controller 1220. The DVD drive 1226 may be a DVD-ROM drive, a DVD-RAM drive, or the like. The storage device 1224 may be a hard disk drive, a solid state drive, or the like. The computer 1200 also includes a ROM1230 and legacy input/output units such as a keyboard, which are connected to the input/output controller 1220 via an input/output chip 1240.

The CPU 1212 operates according to programs stored in the ROM 1230 and the RAM 1214, thereby controlling each of the units. The graphics controller 1216 obtains image data generated by the CPU 1212 in a frame buffer or the like provided in the RAM 1214 or itself, and causes the image data to be displayed on a display device 1218.

The communication interface 1222 communicates with other electronic devices via a network. The storage device 1224 stores programs and data used by the CPU 1212 in the computer 1200. The DVD drive 1226 reads a program or data from the DVD-ROM 1227 or the like and provides the program or data to the storage device 1224. The IC card drive reads the program and data from the IC card and/or writes the program and data to the IC card.

The ROM 1230 stores therein a boot program executed by the computer 1200 at the time of activation and/or a program depending on hardware of the computer 1200. The input/output chip 1240 may also connect various input/output units to the input/output controller 1220 via a USB port, a parallel port, a serial port, a keyboard port, a mouse port, or the like.

Programs are provided by a computer-readable storage medium such as the DVD-ROM 1227 or an IC card. The programs are read from a computer-readable storage medium, installed in the storage device 1224, the RAM 1214, or the ROM 1230, which is also an example of a computer-readable storage medium, and executed by the CPU 1212. Information processing described in those programs is read by the computer 1200 and brings about cooperation between the programs and the various types of hardware resources. A device or a method may be configured by implementing an operation or processing of information according to use of the computer 1200.

For example, in a case in which communication is performed between the computer 1200 and an external device, the CPU 1212 may execute a communication program loaded in the RAM 1214 and instruct the communication interface 1222 to perform communication processing based on processing described in the communication program. Under control of the CPU 1212, the communication interface 1222 reads transmission data stored in a transmission buffer area provided in a recording medium such as the RAM 1214, the storage device 1224, the DVD-ROM 1227, or the IC card, transmits the read transmission data to the network, or writes reception data received from the network to a reception buffer area or the like provided on the recording medium.

In addition, the CPU 1212 may cause the RAM 1214 to read all or a necessary portion of a file or database stored in an external recording medium such as the storage device 1224, the DVD drive 1226 (DVD-ROM 1227), an IC card, or the like, and may execute various types of processing on data on the RAM 1214. Next, the CPU 1212 may write back the processed data to the external recording medium.

Various types of information such as various types of programs, data, tables, and databases may be stored in a recording medium and subjected to information processing. The CPU 1212 may execute various types of processing on the data read from the RAM 1214, including various types of operations, information processing, condition determination, conditional branching, unconditional branching, information search/replacement, and the like, which are described throughout the disclosure and specified in command sequences of a program, and writes back the results to the RAM 1214. In addition, the CPU 1212 may search for information in a file, a database, or the like in the recording medium. For example, in a case in which multiple entries each having an attribute value of a first attribute associated with an attribute value of a second attribute are stored in the recording medium, the CPU 1212 may search for an entry with the attribute value of the first attribute matching the specified condition from the multiple entries, read the attribute value of the second attribute stored in the entry, and thereby acquire the attribute value of the second attribute associated with the first attribute satisfying a predetermined condition.

The programs or software modules described above may be stored in a computer-readable storage medium on or near the computer 1200. Furthermore, a recording medium such as a hard disk or a RAM provided in a server system connected to a dedicated communication network or the Internet can be used as a computer-readable storage medium, thereby providing a program to the computer 1200 via the network.

The blocks in the flowcharts and block diagrams in the present embodiment may represent stages of a process in which an operation is performed or “units” of a device that are responsible for performing the operation. Certain stages and “units” may be implemented by a dedicated circuit, a programmable circuit provided with computer-readable instructions stored on a computer-readable storage medium, and/or a processor provided with computer-readable instructions stored on a computer-readable storage medium. The dedicated circuit may include a digital and/or analog hardware circuit, and may include an integrated circuit (IC) and/or a discrete circuit. The programmable circuit may include a reconfigurable hardware circuit including, for example, logical AND, logical OR, exclusive OR, NAND, NOR, and other logical operations, flip-flops, registers, and memory elements, such as a field programmable gate array (FPGA) and a programmable logic array (PLA).

A computer-readable storage medium may include any tangible device capable of storing instructions to be executed by a suitable device, such that a computer-readable storage medium having instructions stored thereon will comprise an article of manufacture including instructions that, when executed, create means for performing the operations specified in the flowcharts or block diagrams. Examples of the computer-readable storage medium may include an electronic storage medium, a magnetic storage medium, an optical storage medium, an electromagnetic storage medium, a semiconductor storage medium, and the like. More specific examples of the computer-readable storage medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a Blu-Ray (registered trademark) disk, a memory stick, an integrated circuit card, and the like.

The computer-readable instructions may include any of source codes or object codes written in any combination of one or more programming languages, including assembler instructions, instruction-set-architecture (ISA) instructions, machine instructions, machine-dependent instructions, microcode, firmware instructions, state-setting data, or an object-oriented programming language such as Smalltalk, JAVA (registered trademark), C++, or the like, and conventional procedural programming languages, such as the ‘C’ programming language or similar programming languages.

The computer readable instructions may be provided to processors of general purpose computers, special purpose computers, or other programmable data processing devices, or programmable circuits, either locally or over a wide area network (WAN), such as a local area network (LAN), the Internet, or the like, to cause the processors or programmable circuits of the general purpose computers, special purpose computers, or other programmable data processing devices to execute the computer readable instructions to generate means for the processors or programmable circuits to perform the operations specified in the flowcharts or block diagrams. Examples of the processor include a computer processor, a processing unit, a microprocessor, a digital signal processor, a controller, a microcontroller, and the like.

Although the invention has been described with reference to the embodiments above, the technical scope of the invention is not limited to the scope described in the embodiments. It is apparent to those skilled in the art that various modifications or improvements can be made to the above embodiments. It is apparent from the description of the claims that a mode to which such modifications or improvements are added can also be included in the technical scope of the invention.

It should be noted that the order of execution of each processing such as operations, procedures, steps, and stages in the devices, systems, programs, and methods shown in the claims, the specification, and the drawings can be realized in any order unless “before”, “prior to”, or the like is explicitly stated, and unless the output of the previous processing is used in the later processing. Even if the operation flow in the claims, the specification, and the drawings is described using “first,”, “next,”, and the like for convenience, it does not mean that it is essential to perform in this order.

1. An interactive robotic apparatus comprising:
a housing defining an external form factor of the robotic apparatus, the housing including a fabric material forming an outer surface;
a motor assembly mounted within the housing, the motor assembly including a plurality of servo motors configured to produce physical gestures of the robotic apparatus;
a speaker mounted within the housing;
a sensor array including:
a microphone configured to capture audio signals including utterances of a user, and
a camera configured to capture image frames of the user;
a storage device storing a behavior determination model and health parameter data associated with the user, the health parameter data including at least one of body temperature, heart rate, blood pressure, blood glucose level, sleep duration, respiratory rate, or trembling; and
circuitry configured to:
recognize a health state of the user based on the health parameter data,
determine, based on the health state, that the user has a health concern,
generate health advice for the user by applying the behavior determination model to the health state, and
control the speaker to output an utterance of an avatar providing the health advice to the user while controlling the motor assembly to produce a physical gesture accompanying the utterance.

2. The apparatus of claim 1, wherein the circuitry is further configured to detect, from the audio signals captured by the microphone, an utterance by the user indicative of a health concern.

3. The apparatus of claim 1, wherein the health advice includes a medication recommendation generated by the circuitry based on the health state.

4. The apparatus of claim 3, wherein the circuitry is further configured to autonomously determine a symptom of the user based on the health parameter data, and wherein the medication recommendation is generated based on the determined symptom.

5. The apparatus of claim 1, wherein the health parameter data includes a blood pressure of the user acquired via wireless communication with a wearable device worn by the user.

6. The apparatus of claim 1, wherein the health parameter data includes a blood glucose level of the user acquired via wireless communication with a wearable device worn by the user.

7. The apparatus of claim 1, wherein the health parameter data includes a sleep duration of the user stored in time series in the storage device.

8. The apparatus of claim 1, wherein the health parameter data includes a respiratory rate of the user detected by the sensor array.

9. The apparatus of claim 1, wherein the health parameter data includes trembling of a hand of the user detected from the image frames captured by the camera.

10. The apparatus of claim 1, wherein the circuitry is further configured to analyze a complexion of the user from the image frames captured by the camera to determine the health state.

11. The apparatus of claim 1, wherein the circuitry is further configured to analyze an inflection of a conversation of the user from the audio signals captured by the microphone to determine the health state.

12. The apparatus of claim 1, wherein the sensor array further includes a thermo sensor configured to measure the body temperature of the user.

13. The apparatus of claim 1, wherein the behavior determination model comprises a text generation model, and wherein the circuitry is configured to generate the health advice by inputting the health parameter data to the text generation model and obtaining an output representing the health advice.

14. The apparatus of claim 1, wherein the circuitry is further configured to autonomously and periodically check the health condition of the user to watch over the user.

15. The apparatus of claim 1, wherein the utterance of the avatar includes words expressing concern for the health condition of the user.

16. The apparatus of claim 1, wherein the circuitry is further configured to determine an emotion value of the user based on the audio signals captured by the microphone, and wherein the physical gesture accompanying the utterance is determined based on the emotion value.

17. The apparatus of claim 1, wherein the fabric material comprises a cloth fabric forming a soft outer surface of the housing.

18. An interactive robotic apparatus comprising:
a housing formed as a stuffed toy having a shape of an animal and covered with a cloth fabric forming an outer surface, the housing defining a space portion therein;
a motor assembly disposed within the space portion, the motor assembly including motors configured to drive an arm, a hand, and a foot of the stuffed toy to produce physical gestures;
a speaker disposed at a portion corresponding to a mouth of the stuffed toy;
a microphone disposed at a portion corresponding to an ear of the stuffed toy, the microphone configured to capture audio signals including utterances of a user;
a camera disposed at a portion corresponding to an eye of the stuffed toy, the camera configured to capture image frames of the user;
a storage device storing a text generation model and health parameter data associated with the user, the health parameter data including at least one of body temperature, heart rate, blood pressure, blood glucose level, sleep duration, respiratory rate, or trembling; and circuitry configured to:
autonomously and periodically check a health condition of the user based on the health parameter data,
determine that the user has a health concern based on the health condition,
input the health parameter data to the text generation model to generate health advice for the user,
control the speaker to output an utterance of an avatar providing the health advice to the user, and
control the motor assembly to produce a physical gesture accompanying the utterance.

19. The apparatus of claim 18, wherein the circuitry is further configured to spontaneously determine a symptom of the user based on the health parameter data and to generate a medication recommendation based on the determined symptom, and wherein the utterance includes the medication recommendation.

20. A method performed by an interactive robotic apparatus having a housing including a fabric material and a motor assembly, the method comprising:
capturing, by a microphone mounted within the housing, audio signals including utterances of a user;
capturing, by a camera mounted within the housing, image frames of the user;
storing, by circuitry, health parameter data associated with the user in a storage device, the health parameter data including at least one of body temperature, heart rate, blood pressure, blood glucose level, sleep duration, respiratory rate, or trembling;
recognizing, by the circuitry, a health state of the user based on the health parameter data;
determining, by the circuitry, based on the health state, that the user has a health concern;
generating, by the circuitry, health advice for the user by applying a behavior determination model stored in the storage device to the health state;
outputting, by a speaker mounted within the housing, an utterance of an avatar providing the health advice to the user; and
producing, by the motor assembly, a physical gesture accompanying the utterance.
